SEQUENCE LISTING (2 sequences)

SEQ ID NO: 1 - DNA, 20 bp, Artificial Sequence
  Synthetic
agagtttgatcctggctcag

SEQ ID NO: 2 - DNA, 17 bp, Artificial Sequence
  Synthetic
attaccgcggctgctgg

(19) United States
(12) Patent Application Publication
(10) Pub. No.: US 2023/0381104 A1
(43) Pub. Date: Nov. 30, 2023

(54) COMPOSITIONS AND METHODS FOR MODULATION OF BACTERIAL GENE EXPRESSION

(71) Applicant: University of Florida Research Foundation, Incorporated, Gainesville, FL (US)

(72) Inventors: Christian Jobin, Gainesville, FL (US); Thomas D. Schmittgen, Gainesville, FL (US); Rachel Newsome, Gainesville, FL (US)

(73) Assignee: University of Florida Research Foundation, Incorporated, Gainesville, FL (US)

(21) Appl. No.: 18/026,389

(22) PCT Filed: Sep. 15, 2021
(86) PCT No.: PCT/US2021/050428

§ 371 Date: Mar. 15, 2023

Related U.S. Application Data
(60) Provisional application No. 63/079,159, filed on Sep. 16, 2020.

(51) Int. Cl. A61K 9/127; C12N 15/113; C12N 15/88; C12N 5/09; A61K 31/7105; A61P 31/04

(52) U.S. Cl. CPC A61K 9/127; C12N 15/113; C12N 15/88; C12N 5/0693; A61K 31/7105; A61P 31/04; C12N 2310/14; C12N 2320/32

(57) ABSTRACT

Aspects of the disclosure relate to compositions and methods for delivering inhibitory nucleic acids to prokaryotic cells using extracellular vesicles (e.g., exosomes, microvesicles, etc.) derived from mammalian cells. The disclosure provides methods for regulating the expression of one or more genes in a prokaryotic cell. The disclosure further provides methods of treating diseases associated with prokaryotic gene dysregulation.

### RELATED APPLICATION

This application claims the benefit under 35 U.S.C. 119(e) of the filing date of U.S. provisional patent application No. 63/079,159, filed Sep. 16, 2020, entitled “COMPOSITIONS AND METHODS FOR MODULATION OF BACTERIAL GENE EXPRESSION”, the entire contents of which are incorporated herein by reference.

### GOVERNMENT SUPPORT

This invention was made with government support under grant numbers: R01 DK073338 and R21 AI164741 awarded by the National Institutes of Health. The government has certain rights in the inventions.

### BACKGROUND

The microbiota, defined as a community of microorganisms, is considered an important modulator of human health and disease. Humans acquire this microbiota at birth and maintain the ecosystem until death. Among the various microbiota present in the host, the intestinal microbiota is the richest and most complex ecosystem. The largest group of microorganisms forming the intestinal microbiota are bacteria, whose numbers range in the trillions. This microbiota provides a myriad of beneficial functions for the host, including education of the immune system, synthesis of essential vitamins, generation of various nutrients from complex dietary carbohydrates, and ecological competition to fend off invading pathogenic microbes. Because the microbiota is sensitive to environmental modifications such as stress, inflammation, diet, and medications, it is thought that these changes could promote diseases. Indeed, a number of scientific studies have reported changes in the make up or composition of microbiota in diseases such as metabolic syndrome, inflammatory bowel diseases, hypertension, asthma, cardiovascular diseases, rheumatoid arthritis and various forms of cancer. Some of these links have been shown to be causative factors of diseases by using specific bacteria in pre-clinical models.

The biggest hindrance for the field of microbiota is the inability to functionally identify bacterial genes responsible for a given phenotype. Indeed, more than 90% of bacteria forming the microbiota are not amenable to genetic manipulation by available techniques such as Flp-FRT recombinase, transposon mutagenesis, or chemical screens. This limitation is a severe impediment for conducting mechanistic studies to understand the role of microbiota in disease, which in turn hinders the development of therapeutic modalities. For example, targeting microbial genes responsible for toxin production, resistance to antibiotics, or virulence phenotypes would have a tremendous impact in medicine. Therefore, there is a need to develop new tools to alter bacterial genomes.

### SUMMARY

Aspects of the disclosure relate to compositions and methods for delivering one or more inhibitory nucleic acids to prokaryotic cells. The disclosure provides extracellular vesicles (EVs) comprising one or more inhibitory nucleic acids that target one or more genes in a prokaryotic cell, wherein the EVs are derived from eukaryotic cells (e.g., mammalian cells, plant cells, etc.), methods of producing the EVs, and associated methods of use. The EVs of the disclosure may be used to modulate the expression of one or more genes in a prokaryotic cell in vitro or in vivo within a subject. The disclosure is based, in part, on the surprising discovery by the inventors that EVs derived from mammalian cells can be loaded with inhibitory nucleic acids targeting prokaryotic genes and that such inhibitory nucleic acids are properly processed to produce active siRNA molecules (e.g., “guide strands”) both by the EVs and in bacterial cells. In some embodiments, the EVs further comprise mammalian machinery required for the processing and/or functioning of the inhibitory nucleic acids.

In one aspect, the disclosure provides a method of delivering one or more inhibitory nucleic acids to a prokaryotic cell, comprising contacting the prokaryotic cell with an extracellular vesicle (EV) comprising the one or more inhibitory nucleic acids, wherein the one or more inhibitory nucleic acids target (e.g., hybridize to) one or more genes in the prokaryotic cell, and wherein the EV is derived from a mammalian cell.

In one aspect, the disclosure provides a method of regulating the expression of one or more genes in a prokaryotic cell, comprising contacting the prokaryotic cell with an extracellular vesicle (EV) comprising one or more inhibitory nucleic acids that target the one or more genes, wherein the EV is derived from a mammalian cell.

In one aspect, the disclosure provides a method of treating a disease in a subject in need thereof, comprising administering to the subject isolated extracellular vesicles (EVs) comprising one or more inhibitory nucleic acids that target one or more genes in a prokaryotic cell, and wherein the isolated EVs are derived from a mammalian cell. In some embodiments, the subject is human. In some embodiments, the disease is a metabolic disorder, a cardiovascular disease, cancer, an autoimmune disease, or an inflammatory disease. In some embodiments, the disease is metabolic syndrome, inflammatory bowel disease, hypertension, asthma, diabetes, celiac disease, obesity, non-alcoholic fatty liver disease, non-alcoholic steatohepatitis, or rheumatoid arthritis. In some embodiments, the disease is a cancer selected from: lymphoma, leukemia, multiple myeloma, breast cancer, prostate cancer, esophageal cancer, stomach cancer, colorectal cancer, liver cancer, cervical cancer, ovarian or uterine cancer, pancreatic cancer, lung cancer, brain cancer, sarcoma, and skin cancer. In some embodiments, the disease is a bacterial infection.

In one aspect, the disclosure provides a method of modifying the composition of the microbiota in a subject, comprising administering to the subject isolated extracellular vesicles (EVs) comprising one or more inhibitory nucleic acids that target one or more genes in a prokaryotic cell, and wherein the isolated EVs are derived from a mammalian cell. In some embodiments, the method comprises killing prokaryotic cells of a species. In some embodiments, the subject is human. In some embodiments, the microbiota is gastrointestinal microbiota, mucosal microbiota, skin microbiota, microbiota of the respiratory system, microbiota of the ear, nose, and throat, oral microbiota, or microbiota of the urinary tract.

In one aspect, the disclosure provides a method of preparing extracellular vesicles (EVs) derived from a mammalian cell, comprising: (a) introducing one or more nucleic acids comprising or encoding one or more inhibitory nucleic acids that target one or more prokaryotic genes, into a mammalian cell; (b) culturing the mammalian cell under conditions under which the mammalian cell produces EVs; and (c) isolating the EVs from the mammalian cell. In some embodiments, the method further comprising purifying the EVs. In some embodiments, the one or more nucleic acids encoding or comprising the one or more inhibitory nucleic acids are introduced by electroporation, transfection, gene gun, direct injection, microinjection, nucleofection, lipofection, or high-pressure spraying.

In one aspect, the disclosure provides a prokaryotic cell comprising an inhibitory nucleic acid derived from a mammalian cell and one or more components of an RNA-induced silencing complex (RISC). In some embodiments, the prokaryotic cell further comprises one or more of protein kinase RNA activator (PACT), transactivation response RNA binding protein (TRBP), Dicer, Argonaute, Drosha, and Pasha. In some embodiments, the inhibitory nucleic acid and the one or more components of a RISC are present within an extracellular vesicle (EV) derived from a mammalian cell.

In some embodiments, the EV is an exosome or microvesicle. In some embodiments, the EV is an exosome. In some embodiments, an exosome comprises one or more polypeptides selected from: Alix, TSG101, CD9, CD63, CD81, CD82, Flotillin-1, CD24, HSC70, HSP90, ACTB, GAPDH, ENO1, YWHAZ, and PKM2.

In some embodiments, the one or more inhibitory nucleic acids are small interfering RNA (siRNA), microRNA (miRNA), short hairpin RNA (shRNA), antisense RNA, dsRNA, artificial miRNA, circular RNA, long non-coding RNA (lncRNA), or piwi-interacting RNA (piRNA) molecules. In some embodiments, the one or more inhibitory nucleic acids are siRNA molecules. In some embodiments, the one or more inhibitory nucleic acids are miRNA molecules.

In some embodiments, the EV further comprises one or more components of an RNA-induced silencing complex (RISC). In some embodiments, the EV further comprises one or more of protein kinase RNA activator (PACT), transactivation response RNA binding protein (TRBP), Dicer, Argonaute, Drosha, and Pasha.

In some embodiments, the target gene is located chromosomally. The method of any preceding claim, wherein the target gene is located episomally.

In some embodiments, the prokaryotic cell is a bacterial cell. In some embodiments, the bacterial cell is a pathogenic bacterium. In some embodiments, the bacterial cell is a commensal bacterium. In some embodiments, the bacterial cell is a cell type found in human microbiota. In some embodiments, the bacterial cell is of the genus Actinomyces, Akkermansia, Alistipes, Anaerofilum, Anaerostipes, Bacteroides, Barnesiella, Bifidobacterium, Blautia, Campylobacter, Catabacter, Clostridium, Coprobacillus, Enterobacter, Enterococcus, Erysipelotrichaceae, Escherichia, Eubacterium, Faecalibacterium, Flavinofractor, Flavobacterium, Fusobacterium, Gordonibacter, Haemophilus, Holdemania, Hungatella, Lachnospiracea, Lactobacillus, Parabacteroides, Phascolarctobacterium, Prevotella, Pseudomonas, Robinsoniella, Romboutsia, Roseburia, Ruminococcus, Salmonella, Shigella, or Terrisporobacter.

In some embodiments, the mammalian cell is an amniocyte cell, a cardiac progenitor cell, a cardiomyocyte, an epidermal cell, an epithelial cell, a fibroblast, a hematopoietic stem cell, a mesenchymal stem cell, a neuronal precursor cell, a neuron, a platelet, or a reticulocyte. In some embodiments, the mesenchymal stem cell is derived from adipocytes, neurons, bone marrow, or umbilical cord. In some embodiments, the mammalian cell is a cell line selected from: HEK-293, HEK-293T, CHO, PERC6, BJ, fHDF/TERT166, AGE1.HN, CAP, and RPTEC/TERT1.

In some embodiments, the mammalian cell is a cancer cell. In some embodiments, the cancer cell is a bladder cancer cell line, a breast cancer cell line, a brain cancer cell line, a colorectal cancer cell line, a head and neck cancer cell line, a leukemia cell line, a liver cancer cell line, a lung cancer cell line, a lymphoma cell line, an ovarian cancer cell line, a pancreatic cancer cell line, a sarcoma cell line, a stomach cancer cell line, or a uterine cancer cell line. In some embodiments, the cancer cell is a cell line selected from: HT-1080, HeLa, HT29, HTC116, MBA-MB-231, MCF7, Panc-1, OVCAR-4, SW620, KM12, Colo205, and HCT-15.

In some embodiments, the one or more target genes in the prokaryotic cell encode a gene product associated with antibiotic resistance, virulence, biofilm formation, stress response, protein export, bacterial secretion, amino acid biosynthesis, metabolic pathways, flagellar assembly, carbon fixation, adhesion, or iron acquisition. In some embodiments, the one or more target genes in the prokaryotic cell encode a toxin, an adhesin, a receptor, a membrane protein, a structural protein, or a secreted protein.

In one aspect, the disclosure provides a composition comprising EVs prepared according to the methods of the disclosure.

In one aspect, the disclosure provides a prokaryotic cell comprising EVs prepared according to the methods of the disclosure. In some embodiments, the prokaryotic cell is in a subject or in an organ of a subject. In some embodiments, the prokaryotic cell is a bacterial cell.

### BRIEF DESCRIPTION OF DRAWINGS

FIGS. 1A-1D depict the characterization of extracellular vesicles (EVs) produced from HCT116 cells. FIG. 1A shows the particle size and concentration of EVs as determined using Nanotracking analysis. FIG. 1B shows the morphology of EVs as determined using cryo transmission electron microscopy. FIG. 1C shows a Western blot analysis of immunoprecipitated (IP) protein, input, or supernatant from HCT116 cells or HCT116 EVs using antibodies for Ago2 (upper) or GAPDH (lower). FIG. 1D shows the results of qPCR analysis of siRNA levels in HCT116 cells or HCT116 EVs. Data are presented as 2-ACT.

FIG. 2 is a schematic depicting the workflow of microbial gene silencing using EV-mediated siRNA delivery into bacteria, in one embodiment.

FIG. 3 is a graph showing the effect of exposure of E. coli cells stably expressing a gentamicin resistance gene to EVs loaded with an siRNA that specifically targets the gentamicin resistance gene or with a control non-specific (scramble) siRNA, on cell growth on gentamicin-supplemented media.

FIG. 4 is a Western blot analysis of GFP expression after exposure of E. coli cells stably expressing the gfpmut3 plasmid to EVs loaded with an siRNA that specifically targets the gfpmut3 gene or with a control non-specific (scramble) siRNA.

FIGS. 5A-5B show the experimental design and microbial community gene expression associated with biofilm status. FIG. 5A is a schematic showing the setup of the gnotobiotic association (I) and reassociation (III) experiments, along with the analyses done on the stool and tissue samples (II and IV) at the end of the 12-week experiments. Twelve-week stool and/or DC tissue samples were used for RNA, miRNA, and 16S rRNA sequencing analyses (II). Tissue was collected from 12-week-associated BF-bx mice and 16- to 20-week-associated BF+T mice to make the reassociation inoculums (III). FIG. 5B shows principal-component analysis (PCA) of bacterial transcriptomes from BF+T- and BF-bx-associated Apc^{MinΔ850/+};Il10^{-/-} mice generated from Trinity de novo assembly (N=5 for BF+T and BF-bx).

FIGS. 6A-6B show mouse gene expression affected by biofilm status. FIG. 6A shows PCA of mouse transcriptomes from BF+T and BF-bx mice (N=5 per group). FIG. 6B shows the PPAR signaling pathway depicting genes significantly increased in BF+T associated mice in red. Boxes without color denote no significant change.

FIGS. 7A-7B show fecal miRNA profiles and significantly differentially expressed (DE) miRNAs according to biofilm status. FIG. 7A shows a PCA plot of log-normalized mature miRNA counts in GF, BF-bx, and BF+T Apc^{MinΔ850/+};Il10^{-/-} stool (N=4, 7, and 10 for GF, BF-bx, and BF+T, respectively). Symbols represent miRNAs from individual mice for BF-bx and BF+T groups. For the GF group, symbols represent miRNAs pooled from 2 to 5 mice (13 mice total). FIG. 7B shows significant (P_{FDR}<0.05) DE miRNA between the three experimental groups. BF+T microbiota elicited more host miRNAs than the GF and BF-bx microbiota with six miRNAs uniquely DE in response to it. The arrows next to miRNA names indicate whether the miRNA is increased (1) or decreased (2) in the first group listed relative to the second for each comparison. Arrows indicate direction of miRNA expression in BF+T mice compared to BF-bx mice, direction of miRNA expression in BF+T mice compared to GF mice, direction of miRNA expression in BF-bx mice compared to GF mice. Arrows having the same direction indicate miRNAs that are shared between comparison groups and go in the same direction. The miRNAs that are significantly correlated with taxa identified from 16S rRNA sequencing of the stool are highlighted, those that significantly correlated with taxa identified from the DC tissue are highlighted, and those correlating with both are in black font and underlined (total of 11 miRNAs).

FIGS. 8A-8D show that microbial relative abundance at the genus level correlates with specific miRNAs. FIGS. 8A-8B are heatmaps depicting the mean log₁₀-normalized relative abundance of genera within the stool (FIG. 8A) or distal colon tissue (FIG. 8B) compartments that have significant (P_{FDR}<0.05) correlation with miRNA expression. The name of each miRNA is shown below the genus it correlates with. Genera in light font are significantly different between BF-bx and BF+T Apc^{MinΔ850/+};Il10^{-/-} mice. The underlined genera were significantly different based on biofilm status in both the initial association and reassociation experiments. The direction of correlation is shown within parentheses. Relative abundance data are from the subset of BF-bx and BF+T mice that were used for miRNA sequencing (n=7 for BF-bx; n=10 for BF+T). FIG. 8C shows a heatmap comparing the log 2-transformed number of predicted bacterial versus mouse gene targets for the miRNAs that were significantly DE between the GF, BF-bx, or BF+T group. There is a significant negative correlation (Pearson) between the number of predicted bacterial versus mouse gene targets for the set of significant DE miRNAs. FIG. 8D is a scatter plot demonstrating the significant negative Pearson correlation between the log 2-transformed number of mouse versus bacterial gene targets where each circle represents a unique mouse miRNA that was significantly DE between GF, BF-bx, or BF+T group.

FIGS. 9A-9C show biofilm status is associated with microbiota changes in reassociated Apc^{MinΔ850/+};Il10^{-/-} mice. FIGS. 9A-9B show principal-coordinate analyses (PCoAs) of BF-bx and BF+T reassociated mice, with the composition of the stool and DC tissue compartment on the left and right, respectively (N=9, 7, and 5 for BF-bx DC, BF+T PC, and BF+T DC, respectively). (FIG. 9A) BF+T PC reassociation compared to the BF-bx DC reassociation. (FIG. 9B) BF+T DC reassociation compared to the BF-bx DC reassociation. FIG. 9C is a heatmap depicting the mean log₁₀-normalized relative abundances of genera that were significantly different in the stool and/or DC tissues of reassociated mice inoculated with murine colon tissue homogenates derived from human BF+T or BF-bx tissue-associated mice. The underlined genera were significantly different based on biofilm status in both the initial association and the reassociation, and the underlines represent the three genera that correlated with specific miRNAs (FIGS. 8A-8B).

FIG. 10 is a schematic depiction of the major findings. The core, transmissible bacteria found in biofilm-positive tumor (BF+T) associated and reassociated mice are listed under core bacteriome. Some of the bacterial and mouse genes that were differentially expressed in the BF+T associated mice compared to biofilm-negative (BF-bx) associated mice are listed. Fecal miRNAs were also differentially expressed according to biofilm status, correlated with the relative abundances of some bacterial taxa, and were predicted to target mouse and bacterial genes.

FIG. 11A shows that PCA of bacterial transcriptomes from BF+T- and BF-bx-associated Apc^{MinΔ850/+}; Il10^{-/-} mice generated from aligning the reads to the human gut microbiome integrated gene catalog (IGC) show similar clustering to that obtained from de novo assembly (N=5 for BF+T and BF-bx). Bacterial pathways increased in BF+T mice. FIGS. 11B-11C show a visualization of significant DE genes within the bacterial secretion system (as shown in FIG. 11B) and flagellar assembly (as shown in FIG. 11C). KEGG pathways created with Pathview. Genes that were increased in BF+T mice are depicted in (asterisks (*)), while decreased genes are depicted in (plus sign (+)).

FIGS. 12A-12C show that miRNA expression correlation with tumor numbers and miRNA correlation with mouse and bacterial gene targets are not due to chance. This figure is related to FIG. 8. FIG. 12A shows Spearman's rank correlation with P_{FDR}. Three BF-bx samples are at the 0,0 coordinates and are represented as one circle; all other circles represent individual mice. N=5 and 10 for BF-bx and BF+T, respectively. Two BF-bx samples were excluded due to colon collection method. Mmu-miR-140-3p also correlates with Lachnospiraceae incertae sedis abundance in the stool compartment (see FIG. 8). FIGS. 12B-12C show correlating the number of predicted bacterial versus mouse gene targets for 100 randomly selected sets of 25 mouse miRNAs from the list of 127 miRNAs that were detected in at least 25% of the samples (excluding those shown in FIG. 8C) show no significant correlations. FIG. 12B is a boxplot showing the correlation coefficients and FIG. 12C is a boxplot showing −log₁₀ P values for the correlation in FIG. 12B. The dotted line marks P=0.05, values above the line indicate P<0.05, and values below the line indicate P>0.05.

FIGS. 13A-13D show that bacterial composition of biofilm-negative reassociated mice resembles the initial biofilm-negative association. This figure is related to FIGS. 9A-9C. FIGS. 13A-9B show PCoAs comparing stool (as shown in FIG. 13A) and DC tissue (as shown in FIG. 13B) bacterial composition between the BF-bx mice and BF-bx DC reassociated mice. FIG. 13C shows operational taxonomic unit (OTU) level PCoA of the BF-bx mice and BF-bx DC reassociated mice stools generated from rarefied QIIME closed-reference OTUs using unweighted UniFrac distance metric. FIG. 13D shows suboperational taxonomic unit (sOTU) level PCoA of the BF-bx mice and BF-bx DC reassociated mice stools generated from rarefied Deblur sOTUs using unweighted UniFrac distance metric. For stool bacterial composition (FIG. 13A), N=7 and 9 for BF-bx and BF-bx DC, respectively. For DC tissue bacterial composition (FIGS. 13A-13D), N=6 and 9 for BF-bx and BF-bx DC, respectively. The BF-bx DC reassociation inoculum was prepared from additional DC tissue pieces taken from four of the BF-bx-associated mice.

FIGS. 14A-14D show that bacterial composition of biofilm-positive reassociated mice shifts compared to the initial biofilm-positive association, but the majority of candidate biofilm-associated taxa were unaffected. This figure is related to FIGS. 9A-9C and FIGS. 15A-15B. FIGS. 14A-14B show PCoAs comparing stool (as seen in FIG. 14A) and DC tissue (as seen in FIG. 14B) bacterial composition between the BF+T mice and either the BF+T PC (left panel) or BF+T DC (right panel) reassociated mice (N=7, 7, and 5 for BF+T, BF+T PC, and B+T DC, respectively). The BF+T reassociation inoculums were made from either additional PC or DC tissue pieces from four of the BF+T associated mice. FIGS. 14C-14D show heatmaps representing the mean log₁₀-normalized relative abundances of genera that were significantly different between the initial association and the reassociation in either the stool (as seen in FIG. 14C) or DC tissue (as seen in FIG. 14D) compartments. The underlined genera were significantly different in the reassociated mice based on biofilm status in the initial association.

FIGS. 15A-15B show that biofilm-positive reassociated mice establish similar bacterial communities regardless of the colon region used to make the reassociation inoculums. This figure is related to FIGS. 9A-9C and FIGS. 14A-14D. FIG. 15A shows PCoAs comparing the stool and DC tissue bacterial communities from BF+T reassociated mice given an inoculum made from either proximal (BF+T PC) or distal (BF+T DC) colon tissues (N=7 and 5 for BF+T PC and BF+T DC, respectively). FIG. 15B shows heatmaps depicting the mean log₁₀-normalized relative abundances of genera that were significantly different between the two BF+T reassociation groups. The underlined genera were part of the 24 genera significantly different based on biofilm status in the initial association, but were not maintained in one of the BF+T reassociation groups.

FIGS. 16A-16B show knockdown of an endogenous bacterial gene using EV-loaded siRNA. FIG. 16A shows representative data indicating EV-loaded siRNA targeting E. coli FliC is processed to produce active guide strand both in the EV and in E. coli cells. FIG. 16B shows representative data indicating EV-loaded siRNA mediated target gene (FliC) knockdown relative to scrambled siRNA controls.

### DETAILED DESCRIPTION

The disclosure provides extracellular vesicles (EVs) comprising one or more inhibitory nucleic acids that target one or more genes in a prokaryotic cell, wherein the EVs are derived from mammalian cells, compositions and prokaryotic cells comprising the EVs, methods of producing the EVs, and associated methods of use. The EVs of the disclosure may be used to modulate the expression of one or more genes in a prokaryotic cell in vitro or in vivo within a subject. The compositions and methods of the disclosure are useful to treat and/or prevent various diseases.

### Definitions

General methods in molecular and cellular biochemistry can be found in such textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

The term “extracellular vesicle delivery” or “delivery of extracellular vesicles” refers to the administration and localization of extracellular vesicles to target tissues, cells, and/or organs in vivo, in vitro, or ex vivo.

The terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some embodiments, isolating or purifying as used herein is the process of removing, partially removing (e.g. a fraction) of the EVs from a sample containing producer cells. An “isolated” EV (e.g., exosome, microvesicle, etc.) is one that has been removed from a biological source or from a medium in which producer cells have been cultured. For instance, an isolated EV is an EV that has been removed from a conditioned medium and the other biological components present in the medium, e.g., proteins, polynucleotides, and other cellular material of the medium are no longer present.

The term “mammal” as used herein includes both humans and non-human mammals.

The term “modulate,” “modulating”, “modify,” and/or “modulator” generally refers to the ability to alter, by increase or decrease, e.g., directly or indirectly promoting/stimulating/up-regulating or interfering with/inhibiting/down-regulating a specific concentration, level, expression, function or behavior, such as, e.g., to act as an antagonist or inhibitor, or as an agonist. In some instances, a modulator can increase and/or decrease a certain concentration, level, activity or function relative to a control, or relative to the average level of activity that would generally be expected or relative to a control level of activity. In some embodiments, a control concentration, level, activity, or function is the wild-type concentration level, activity, or function of a gene or gene product (e.g., mRNA, protein, etc.). In some embodiments, a control concentration, level, activity, or function is the concentration, level, activity, or function of a gene or gene product (e.g., mRNA, protein, etc.) prior to contacting with an EV of the disclosure or in the absence of contacting with an EV of the disclosure. In the context of gene expression, “modulate” may be used interchangeably with “regulate.”

The “percent identity” of two nucleic acid sequences may be determined by any method known in the art. In some embodiments, the percent identity of two nucleic acid sequences is determined using the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into the NBLAST and XBLAST programs (version 2.0) of Altschul et al., J. Mol. Biol. 215:403-10, 1990. BLAST nucleotide searches can be performed with the NBLAST program, score=100, wordlength-12, to obtain guide sequences homologous to a target nucleic acid. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

The terms “polypeptide” and “protein” are used interchangeably to refer to a polymer of amino acid residues linked by peptide bonds, and for the purposes of the instant disclosure, have a minimum length of at least 5 amino acids. Both full-length proteins and fragments thereof greater than 5 amino acids are encompassed by the definition. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, as used herein, a “polypeptide” or “protein” refers to a protein that includes modifications, such as deletions, additions, and substitutions (generally conservative in nature as would be known to a person in the art) to the native sequence, as long as the protein maintains the desired activity relevant to the purposes of the described methods. These modifications can be deliberate, as through site-directed mutagenesis, or can be accidental, such as through mutations of hosts that produce the proteins, or errors due to PCR amplification or other recombinant DNA methods.

The terms “nucleic acid” or “nucleic acid molecule,” as used herein, generally refers to a single or double-stranded polymer of deoxyribonucleotide or ribonucleotide bases. The nucleotide monomers in the nucleic acid molecules may be naturally occurring nucleotides, modified nucleotides or combinations thereof. Modified nucleotides, in some embodiments, comprise modifications of the sugar moiety and/or the pyrimidine or purine base.

The terms “subject,” and “patient,” are used interchangeably herein and refer to any mammalian subject for whom treatment or therapy is desired. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The term “sufficient amount” means an amount sufficient to produce a desired effect, e.g., an amount sufficient to modulate the expression of a prokaryotic gene.

A “effective amount” of the compositions of the disclosure generally refers to an amount sufficient to elicit the desired biological response, e.g., ameliorate a symptom of a disease or to reduce or inhibit expression of a target gene. In some embodiments, an effective amount is an amount of an inhibitory nucleic acid (e.g., an EV-loaded inhibitory nucleic acid) that reduces expression of a target gene by about 5%, 10%, 20%, 30%, 40%, 50%, 60%, 70%, 80%, 90%, 95%, 99%, or 100%, relative to expression of the target gene in the subject prior to the administration of the inhibitory nucleic acid, or relative to a positive control subject that has not been treated with the inhibitory nucleic acid. As will be appreciated by those of ordinary skill in this art, the effective amount of an agent described herein may vary depending on such factors as the condition being treated, the mode of administration, and the age, body composition, and health of the subject.

The terms “treat”, “treating”, “treatment”, and “therapy” encompass an action that occurs while a subject is suffering from a condition which reduces the severity of the condition (or a symptom associated with the condition) or retards or slows the progression of the condition (or a symptom associated with the condition).

### Delivery of Inhibitory Nucleic Acids to Prokaryotic Cells

The present disclosure is directed to EVs derived from eukaryotic cells (e.g., mammalian cells), and their use as vehicles to deliver one or more inhibitory nucleic acids and/or the associated mammalian machinery to prokaryotic cells. In some embodiments, the eukaryotic cells are mammalian cells. In some embodiments, the eukaryotic cells are plant cells. The disclosure is based, in part, on the discovery that mammalian-derived inhibitory nucleic acids targeting prokaryotic genes and the endogenous mammalian machinery required for the processing and/or function of the inhibitory nucleic acids can be packaged into EVs derived from mammalian cells. Thus, the present disclosure provides, in some embodiments, mammalian “cell factories” that produce EVs for delivering one or more functional inhibitory nucleic acids and/or the associated mammalian machinery to prokaryotic cells, which lack endogenous RNAi machinery. The “cell factories” described herein thus allow targeted regulation (e.g., gene silencing) of prokaryotic genes using mammalian machinery.

As described herein, in silico computer-based analyses identified the existence of a trans-kingdom communication network between mammals and bacteria whereby mammalian-derived inhibitory RNAs target prokaryotic gene expression. Without wishing to be bound by theory, it was observed that the inhibitory RNAs are processed and loaded into the RNA Induced Silencing Complex (RISC) and incorporated into EVs released from mammalian cells. In some embodiments, an EV can interact with a target prokaryotic cell via membrane fusion and deliver the inhibitory nucleic acids to the cytoplasm of the target cell. In some embodiments, gene expression is silenced or repressed following recognition of its target by the inhibitory RNA. It is further demonstrated herein that specific inhibitory nucleic acids targeting one or more genes in a prokaryotic cell can be introduced into and/or expressed in mammalian cells (e.g., a cancer cell) and EVs (e.g., exosomes) comprising the inhibitory nucleic acids and the associated endogenous mammalian machinery required for the processing and/or functioning of the inhibitory nucleic acids (e.g., RISC) can be purified and added to prokaryotic cells to modulate the expression of specific genes. The compositions and methods of the disclosure make it possible to target prokaryotic genes in vitro or in vivo within a subject. The compositions and methods of the disclosure are thus useful for modulating the expression of disease-causing genes in prokaryotic cells as well for targeting prokaryotic genes for functional studies, which in turn will help glean critical information about the role of prokaryotic cells in various biological processes and diseases.

### Extracellular Vesicles

EVs are membrane enclosed vesicles released by all cells into the extracellular environment. Different types of extracellular vesicles can be identified based on the biogenesis pathway. For example, exosomes are formed by inward budding of late endosomes forming multivesicular bodies (MVB) which then fuse with the plasma membrane of the cell concomitantly releasing the exosomes; microvesicles (also referred to as ectosomes, shedding vesicles, or microparticles) are formed by outward budding of the plasma membrane followed by fission; and when a cell is dying via apoptosis, the cell divides its cellular content in different membrane enclosed vesicles termed apoptotic bodies.

The EVs (e.g., exosomes, microvesicles, etc.) of the disclosure may be derived from any suitable mammalian cell. The mammalian cell from which the EVs are derived may also be referred to herein as a “producer cell.” A producer cell can share a protein, lipid, sugar, or nucleic acid component with the EV. In some embodiments, the mammalian cell is a modified cell (e.g., a mammalian cell into which one or more inhibitory nucleic acids targeting one or more prokaryotic genes have been introduced). In some embodiments, the mammalian cell is a cultured or isolated cell. In some embodiments, the mammalian cell is a cell line. In some embodiments, the mammalian cell is a primary cell. In some embodiments, the mammalian cell is a human cell. In some embodiments, the EVs are derived from an immune cell (e.g., B lymphocytes, T lymphocytes, dendritic cell, mast cells, macrophages, etc.). In some embodiments, the EVs are derived from an amniocyte cell, a cardiac progenitor cell, a cardiomyocyte, an epidermal cell, an epithelial cell (e.g., an intestinal epithelial cell), a fibroblast, a hematopoietic stem cell, a mesenchymal stem cell (e.g., mesenchymal stem cells derived from adipocytes, neurons, bone marrow, or umbilical cord, etc.), a neuronal precursor cell, a neuron, a platelet, or a reticulocyte, etc. In some embodiments, the EVs are derived from a mammalian cell line including but not limited to a human embryonic kidney (HEK) cell line (e.g., HEK-293, HEK-293T, etc.), a Chinese hamster ovary (CHO) cell line, a PERC6 cell line, BJ human foreskin fibroblast cells, a fHDF fibroblast cell line (e.g., fDHF/TERT166), AGE1.HN cells, CAP® cells, RPTEC/TERT1 cells, etc. In some embodiments, the EVs are derived from a cancer cell. In some embodiments, the cancer cell is a bladder cancer cell or cell line, a breast cancer cell or cell line, a brain cancer cell or cell line, a colorectal cancer cell or cell line, a head and neck cancer cell or cell line, a leukemia cell or cell line, a liver cancer cell or cell line, a lung cancer cell or cell line, a lymphoma cell or cell line, an ovarian cancer cell or cell line, a pancreatic cancer cell or cell line, a sarcoma cell or cell line, a stomach cancer cell or cell line, or a uterine cancer cell or cell line. In some embodiments, the EVs are derived from a cancer cell line including but not limited to an HT-1080 cell line, a HeLa cell line, a HT29 cell line, a HCT116 cell line, an MBA-MB-231 cell line, a MCF7 cell line, a Panc-1 cell line, an OVCAR-4 cell line, an SW620 cell line, a KM12 cell line, a Colo205 cell line, or a HCT-15 cell line, etc. In some embodiments, the cancer cell is from a colorectal cell line (e.g., HT29, HCT116, etc.). In some embodiments, the EVs of the disclosure may be further modified to deliver the inhibitory nucleic acids to a specific target. In some embodiments, an EV comprises a targeting moiety (e.g., a polypeptide, a polysaccharide, etc.). In some embodiments, the targeting moiety is capable of targeting the EV to a specific target (e.g., a target such as a metabolite, a polypeptide complex, a cell such as a bacterial cell, or a tissue) that circulates in the circulatory system of the subject, such as the blood, or a target that resides in a tissue. In some embodiments, the targeting moiety is introduced into the producer cell (e.g., an exogenous nucleic acid that encodes a targeting polypeptide is introduced into the producer cell). In some embodiments, the targeting moiety is introduced into the EV directly (e.g., after the EV is isolated from the producer cell). In some embodiments, the targeting moiety is a mucin (e.g., MUC1, MUC3A, MUC3B, MUC4, MUC12, MUC13, MUC15, MUC16, MUC17, MUC20, MUC2, MUC5AC, MUC5B, MUC6, MUC7, MUC8, MUC19, etc.). In some embodiments, the targeting moiety is a mucin-binding protein. In some embodiments, the targeting moiety is on the surface of the EV.

In some embodiments, an EV has a diameter between about 20-1000 nm, such as between about 20-100 nm, 20-200 nm, 20-300 nm, 20-400 nm, 20-500 nm, 20-600 nm, 20-700 nm, 20-800 nm, 20-900 nm, 30-100 nm, 30-200 nm, 30-300 nm, 30-400 nm, 30-500 nm, 30-600 nm, 30-700 nm, 30-800 nm, 30-900 nm, 40-100 nm, 40-200 nm, 40-300 nm, 40-400 nm, 40-500 nm, 40-600 nm, 40-700 nm, 40-800 nm, 40-900 nm, 50-150 nm, 50-500 nm, 50-750 nm, 100-200 nm, 100-500 nm, or 500-1000 nm. The term “diameter” as used herein refers to the longest measurable dimension of an EV.

In some embodiments, an EV is an exosome. An exosome may range from about 30 nm to about 150 nm in diameter but can include membrane particles of similar origin up to about 200 nm. In some embodiments, an exosome has a diameter between about 30-100 nm, between about 30-150 nm, between about 30-200 nm, between about 50-100 nm, between about 50-150 nm, between about 50-200 nm, between about 100-150 nm, or between about 100-200 nm. In some embodiments, an exosome has one or more markers selected from: Alix, TSG101, tetraspanins (e.g., CD9, CD63, CD81, CD82), flotillin-1, CD24, HSC70, HSP90, ACTB, GAPDH, ENO1, YWHAZ, PKM2, etc.

In some embodiments, an EV is a microvesicle. A microvesicle may range from about 100 nm to about 1 μm in diameter. In some embodiments, a microvesicle has a diameter between about 100-200 nm, between about 100-300 nm, between about 100-400 nm, between about 100-500 nm, between about 200-600 nm, between about 300-700 nm, between about 400-800 nm, or between about 500 nm-1 μm. In some embodiments, a microvesicle has one or more markers such as but not limited to an integrin, a selectin, CD40, etc.

In some embodiments, an EV is an apoptotic body. In some embodiments, an apoptotic body has one or more markers such as but not limited to annexin V, phosphatidylserine, etc. In some embodiments, an EV is a fragment of a cell. In some embodiments, an EV is a vesicle derived from a cell by direct or indirect manipulation. In some embodiments, an EV is a vesiculated organelle.

Methods of Producing Extracellular vesicles

In some embodiments, the EVs (e.g., exosomes, microvesicles, etc.) of the disclosure are prepared from mammalian cells into which one or more inhibitory nucleic acids targeting one or more prokaryotic genes have been introduced. In some embodiments, the EVs are prepared from mammalian cells and then loaded with the inhibitory nucleic acids.

In some embodiments, the EVs (e.g., exosomes, microvesicles, etc.) are prepared by culturing the mammalian cells under conditions under which the mammalian cells release EVs into the cell culture medium. Thus, in some embodiments, the methods of the disclosure comprise isolation of EVs from cell culture medium. In some embodiments, the cells may be cultured in a stirred tank bioreactor or a hollow fiber bioreactor. In some embodiments, the methods of the disclosure comprise a step of replacing the culture medium with a low serum medium (e.g., a medium comprising about 2% serum or less, about 1% serum or less, about 0.5% serum or less, about 0.4% serum or less, about 0.3% serum or less, about 0.2% serum or less, about 0.1% serum or less), or serum-free medium to increase the production of EVs. In some embodiments, EV production may be increased by inducing hypoxia. In some embodiments, EVs may be isolated from a tissue or organ explant. In some embodiments, EVs may be isolated from physiological fluids, such as plasma, urine, amniotic fluid and malignant effusions.

EVs (e.g., exosomes, microvesicles, etc.) may be isolated from a source material (e.g., cell culture medium, tissue or organ explants, physiological fluid, etc.) by any suitable method known in the art. Isolation may be based on physical properties, including separation on the basis of electrical charge (e.g., electrophoretic separation), size (e.g., filtration, molecular sieving, etc.), density (e.g., regular or gradient centrifugation or ultracentrifugation), Svedberg constant (e.g., sedimentation with or without external force, etc.). Alternatively, or additionally, isolation can be based on one or more biological or biochemical properties, and include methods that can employ surface markers (e.g., for precipitation, reversible binding to solid phase, FACS separation, specific ligand binding, non-specific ligand binding, affinity purification etc.).

Isolation and enrichment can be done in a general and non-selective manner, typically including serial centrifugation. Alternatively, isolation and enrichment can be done in a more specific and selective manner, such as using EV or producer cell-specific surface markers. For example, specific surface markers can be used in immunoprecipitation, FACS sorting, affinity purification, and magnetic separation with bead-bound ligands.

In some embodiments, the isolation of EVs can involve combinations of methods that include, but are not limited to, size exclusion chromatography, ultracentrifugation, differential centrifugation, size-based membrane filtration, immunoprecipitation, FACS sorting, and magnetic separation.

Other methods include commercially available exosome isolation kits (e.g., Total Exosomes Isolation kit [Life Technologies], Exo-Spin™ Exosome Purification Kit [Cell Guidance Systems], or PureExo® Exosome Isolation Kit [101 Bio]); and commercially available instruments, such as Dynabeads® Human CD63-specific purification system or Dynabeads® Streptavidin purification system (available from Life Technologies Corporation).

The presence, size, and purity, etc. of EVs (e.g., exosomes, microvesicles, etc.) can be characterized by methods such as Western blotting, transmission electron microscopy, cryo electron microscopy, flow cytometry, atomic force microscopy, nanoparticle tracking analysis, Raman microspectroscopy, resistive pulse sensing, transmission electron microscopy, etc.

In some embodiments, an isolated EV (e.g., exosome, microvesicle, etc.) preparation has no detectable undesired activity or, alternatively, the level or amount of the undesired activity is at or below an acceptable level or amount. In other embodiments, an isolated EV preparation has an amount and/or concentration of desired EVs at or above an acceptable amount and/or concentration. In other embodiments, the isolated EV preparation is enriched as compared to the starting material (e.g. producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some embodiments, isolated EV preparations are substantially free of residual biological products. In some embodiments, the isolated EV preparations are 100% free, 99% free, 98% free, 97% free, 96% free, or 95% free of any contaminating biological matter. Residual biological products can include abiotic materials (including chemicals) or unwanted nucleic acids, proteins, lipids, or metabolites. Substantially free of residual biological products can also mean that the isolated EV preparation contains no detectable mammalian producer cells and that only EVs are detectable.

### Inhibitory Nucleic Acids

The EVs (e.g., exosomes, microvesicles, etc.) of the disclosure comprise one or more (e.g., 2 or more, 3 or more, 4 or more, 5 or more, etc.) inhibitory nucleic acids targeting one or more prokaryotic genes. An inhibitory nucleic acid of the disclosure regulates (e.g., inhibits) the expression of a target gene (e.g., by transcriptional gene silencing, posttranscriptional gene silencing, RNAi, etc.) in a sequence-specific manner. In some embodiments, an inhibitory nucleic acid regulates the expression of (e.g., inhibits) only one gene. In some embodiments, an inhibitory nucleic acid regulates the expression of (e.g., inhibits) one gene with higher selectivity and/or specificity relative to other genes (e.g., off-target genes). The inhibitory nucleic acids may be single stranded or double stranded. The inhibitory nucleic acids may be RNA molecules, DNA molecules, or chimeric RNA/DNA molecules. In some embodiments, the inhibitory nucleic acids are dsDNA molecules. In some embodiments, the inhibitory nucleic acids are RNA molecules such as but not limited to small interfering RNA (siRNA), microRNA (miRNA), short hairpin RNA (shRNA), antisense RNA, dsRNA, artificial miRNA, circular RNA, long non-coding RNA (lncRNA), piwi-interacting RNA (piRNA), etc.

An inhibitory nucleic acid of the disclosure comprises a polynucleotide sequence that is at least 65% complementary to a region of the target gene (or target mRNA). The inhibitory nucleic acid comprises a polynucleotide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementarity to a region of the target prokaryotic gene (or target mRNA). In some embodiments, an inhibitory nucleic acid (e.g., a guide strand of an inhibitory nucleic acid) comprises one or more (e.g., 1, 2, 3, 4, 5, 6, 7, 8, 9, 10, or more) mismatches with a target sequence. In some embodiments, one or more of the mismatches forms a wobble-base pair with the target nucleic acid.

In some embodiments, the region of complementarity (in the inhibitory nucleic acid or in the target gene or mRNA) is about 4 to 50 contiguous nucleotides. In some embodiments, the region of complementarity is about 15-30 contiguous nucleotides, about 20-30 contiguous nucleotides, about 20-40 contiguous nucleotides, or about 30-50 contiguous nucleotides, etc. As used herein, the term “complementary” refers to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G), or in any other manner that allows for the formation of stable duplexes. It will be understood that “100% complementarity” refers to the situation in which each nucleotide unit of one polynucleotide strand or region can hydrogen bond with each nucleotide unit of a second polynucleotide strand or region. Less than 100% complementarity refers to the situation in which some, but not all, nucleotide units of two strands or two regions can hydrogen bond with each other. For example, for two 19-mers, if 17 base pairs on each strand or each region can hydrogen bond with each other, the polynucleotide strands exhibit 89.5% complementarity. In some embodiments, an inhibitory nucleic acid may comprise one or more hairpin and/or bulge structures that are non-complementary to the target gene (or target mRNA).

In some embodiments, an inhibitory nucleic acid is a double stranded nucleic acid. In some embodiments, each strand of the inhibitory nucleic acid is about 15-60, 15-50, 15-40 15-30, 15-25, 19-25, 20-30, or 20-24 nucleotides in length. In some embodiments, each strand of the inhibitory nucleic acid is 15, 16, 17, 18, 19, 20, 21, 22, 23, 24, 25, 26, 27, 28, 29, 30, 31, 32, 33, 34, or 35 nucleotides in length. In some embodiments, at least one strand of the inhibitory nucleic acid has a 3′ overhang of 1-5 nucleotides (e.g., 1, 2, 3, 4, or 5 nucleotides). In some embodiments, an inhibitory nucleic acid can also be generated by cleavage of a longer double stranded precursor nucleic acid (e.g., a double stranded precursor nucleic acid greater than about 25 nucleotides in length). In some embodiments, a precursor nucleic acid is at least 50 nucleotides to about 100, 200, 300, 400, or 500 nucleotides in length. A precursor nucleic acid may be as long as 1000, 1500, 2000, 5000 nucleotides in length, or longer.

In some embodiments, an inhibitory nucleic acid is a single stranded nucleic acid. In some embodiments, the inhibitory nucleic acid is about 15-120, 15-60, 15-50, 15-40 15-30, 15-25, 19-25, 20-30, or 20-24 nucleotides in length. In some embodiments, the inhibitory nucleic acid is 15, 16, 17, 18, 19, 20, 21, 22, 23, 24, 25, 26, 27, 28, 29, 30, 31, 32, 33, 34, or 35 nucleotides in length. In some embodiments, an inhibitory nucleic acid can also be generated by cleavage of a longer single stranded precursor nucleic acid. In some embodiments, a single stranded precursor nucleic acid is about 50-150, 60-120, 60-100, or 60-70 nucleotides in length. In some embodiments, a precursor nucleic acid is at least 50 nucleotides to about 100, 200, 300, 400, or 500 nucleotides in length. A precursor nucleic acid may be as long as 1000, 1500, 2000, 5000 nucleotides in length, or longer.

In some embodiments, the inhibitory nucleic acid is processed into its active form within the mammalian cell. In some embodiments, the inhibitory nucleic acid is processed into its active form within the prokaryotic cell.

In some embodiments, the inhibitory nucleic acid or its precursor is introduced directly into a mammalian cell. In some embodiments, the inhibitory nucleic acid or its precursor is introduced directly into an isolated EV. In some embodiments, a DNA molecule encoding (or comprising) the inhibitory nucleic acid or its precursor is introduced into the mammalian cell. In some embodiments, a DNA molecule encoding (or comprising) the inhibitory nucleic acid or its precursor is introduced into an isolated EV. In some embodiments, the DNA molecule encoding (or comprising) the inhibitory nucleic acid or its precursor is operably linked to one or more expression control elements (e.g., promoter and/or enhancer sequences). In some embodiments, the DNA molecule encoding (or comprising) the inhibitory nucleic acid or its precursor is located on a vector (plasmid, viral vector, etc.). Suitable vectors for expressing inhibitory nucleic acids in mammalian cells are known in the art.

In some embodiments, the inhibitory nucleic acid is an RNA molecule. In some embodiments, the RNA molecule or its precursor is introduced directly into the mammalian cell. In some embodiments, the RNA molecule or its precursor is introduced directly into an isolated EV. In some embodiments, a DNA molecule encoding the RNA molecule or its precursor is introduced into the mammalian cell. In some embodiments, a DNA molecule encoding the RNA molecule or its precursor is introduced into an isolated EV. In some embodiments, the DNA molecule encoding the RNA molecule or its precursor is operably linked to one or more expression control elements (e.g., promoter and/or enhancer sequences). In some embodiments, the DNA molecule encoding the RNA molecule or its precursor is located on a vector (plasmid, viral vector, etc.). In some embodiments, the inhibitory nucleic acid (e.g., inhibitory RNA molecule) is introduced directly into the mammalian cell.

Nucleic acids encoding or comprising the one or more inhibitory nucleic acids may be introduced into a mammalian cell by any suitable method known in the art, such as but not limited to, electroporation, transfection, gene gun, direct injection, microinjection, nucleofection, lipofection, high-pressure spraying, etc.

In some embodiments, the inhibitory nucleic acids are RNA molecules that comprise at least one modified nucleotide analog (1 or more, 2 or more, 3 or more, 4 or more, 5 or more, 6 or more, 7 or more, 8 or more, 9 or more, 10 or more, etc.). The nucleotide analogs may be at positions where the activity is not substantially impaired, e.g., at a region at the 5′-end and/or at the 3′-end of the RNA molecule. In some embodiments, overhangs can be stabilized by inserting modified nucleotide analogs. In some embodiments, a modified nucleotide analog comprises a modified nucleobase. In some embodiments, a modified nucleotide analog comprises a modified internucleotide linkage (e.g., a modified phosphate backbone).

In some embodiments, an inhibitory nucleic acid is an siRNA. siRNAs are typically double-stranded RNA molecules. In some embodiments, each strand of the siRNA is about 15-60, 15-50, 15-40 15-30, 15-25, 19-25, 20-30, or 20-24 nucleotides in length. In some embodiments, each strand of the siRNA is 15, 16, 17, 18, 19, 20, 21, 22, 23, 24, 25, 26, 27, 28, 29, 30, 31, 32, 33, 34, or 35 nucleotides in length. In some embodiments, at least one strand of the siRNA has a 3′ overhang of 1-5 nucleotides (e.g., 1, 2, 3, 4, or 5 nucleotides). Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule. In some embodiments, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA. In some embodiments, a dsRNA is at least 50 nucleotides to about 100, 200, 300, 400, or 500 nucleotides in length. A dsRNA may be as long as 1000, 1500, 2000, 5000 nucleotides in length, or longer.

In some embodiments, an inhibitory nucleic acid is an miRNA. In some embodiments, a miRNA may be a single-stranded RNA molecule. In some embodiments, a miRNA may be a double-stranded RNA molecule. In some embodiments, a miRNA is about 15-60, 15-50, 15-40 15-30, 15-25, 19-25, 20-30, or 20-24 nucleotides in length. In some embodiments, a miRNA is 15, 16, 17, 18, 19, 20, 21, 22, 23, 24, 25, 26, 27, 28, 29, 30, 31, 32, 33, 34, or 35 nucleotides in length. In some embodiments, the miRNA is a precursor miRNA (e.g., a pre-miRNA, or a pri-miRNA). In some embodiments, a precursor miRNA is about 50-150, 60-120, 60-100, or 60-70 nucleotides in length. In some embodiments, a precursor miRNA is at least 50 nucleotides to about 100, 200, 300, 400, or 500 nucleotides in length. A precursor miRNA may be as long as 1000, 1500, 2000, 5000 nucleotides in length, or longer.

In some embodiments, an inhibitory nucleic acid is an artificial miRNA (e.g., the desired mature miRNA molecule (e.g., an miRNA targeting a desired prokaryotic gene) is flanked by a different precursor miRNA scaffold). In some embodiments, the precursor miRNA (e.g., pri-miRNA or pre-miRNA) scaffold is derived from mir-30a or miR-155. In some embodiments, the mature miRNA is about 15-60, 15-50, 15-40 15-30, 15-25, 19-25, 20-30, or 20-24 nucleotides in length. In some embodiments, the mature miRNA is 15, 16, 17, 18, 19, 20, 21, 22, 23, 24, 25, 26, 27, 28, 29, 30, 31, 32, 33, 34, or 35 nucleotides in length. In some embodiments, the artificial miRNA is about 50-150, 60-120, 60-100, or 60-70 nucleotides in length. In some embodiments, the artificial miRNA is at least 50 nucleotides to about 100, 200, 300, 400, or 500 nucleotides in length. An artificial miRNA may be as long as 1000, 1500, 2000, 5000 nucleotides in length, or longer.

In some embodiments, the EVs (e.g., exosomes, microvesicles, etc.) of the disclosure further comprise one or more components of the endogenous mammalian machinery required for the processing and/or activity of the inhibitory nucleic acids. In some embodiments, the EVs further comprise one or more components of an RNA induced silencing complex (RISC) (e.g., protein kinase RNA activator (PACT), transactivation response RNA binding protein (TRBP), Dicer, Argonaute, Drosha, Pasha, etc.). In some embodiments, the EVs (e.g., exosomes, microvesicles, etc.) of the disclosure further comprise one or more nucleic acids encoding one or more components of the endogenous mammalian machinery required for the processing and/or activity of the inhibitory nucleic acids. In some embodiments, the EVs further comprise one or more nucleic acids encoding one or more components of an RNA induced silencing complex (RISC) (e.g., protein kinase RNA activator (PACT), transactivation response RNA binding protein (TRBP), Dicer, Argonaute, Drosha, Pasha, etc.).

### Prokaryotic Cells

Any suitable prokaryotic cell can be targeted for delivery of inhibitory nucleic acids via the EVs (e.g., exosomes, microvesicles, etc.) of the present disclosure. A prokaryotic cell may be a bacterial cell or an archaeal cell.

An archaeal cell may be selected from the following phyla: Crenarchaeota, Euryarchaeota, Korarchaeota, Nanoarchaeota, and Thaumarchaeota. In some embodiments, an archaeal cell is a cell type found in human microbiota (e.g., Methanobrevibacter smithii, Methanosphaera stadtmanae, etc.). In some embodiments, the microbiota is gastrointestinal microbiota, mucosal microbiota, skin microbiota, microbiota of the respiratory system, microbiota of the ear, nose, and throat, oral microbiota, or microbiota of the urinary tract.

A bacterial cell may be Gram-negative or Gram-positive. A bacterial cell may be selected from the following phyla: Acidobacteria, Actinobacteria, Aquificae, Armatimonadetes, Bacteroidetes, Campylobacter, Caldiserica, Chlamydiae, Chlorobi, Chloroflexi, Chrysiogenetes, Coprothermobacterota, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospirae, Planctomycetes, Proteobacteria, Spirochaetes, Synergistetes, Tenericutes, Thermodesulfobacteria, Thermotogae, Verrucomicrobia.

In some embodiments, a bacterial cell is a pathogenic bacterium such as but not limited to, Acinetobacter spp. (e.g., A. baumannii); Actinomyces spp. (e.g., A. israelii), Bacillus spp. (e.g., B. anthracis), Bacteroides spp. (e.g., B. fragilis), Bordetalla spp. (e.g., B. pertussis), Borrelia spp. (e.g., B. burgdorferi, B. garinii, B. afzelii, B. recurrentis, etc.), Brucella spp. (B. abortus, B. canis, B. melitensis, B. suis, etc.), Campylobacter spp. (C. jejuni), Chlamydia spp. (C. pneumoniae, C. trachomatis), Chlamydophila spp. (e.g., C. psittaci), Clostridium spp. (e.g., C. botulinum, C. difficile, C. perfringens, C. tetani, etc.), Corynebacterium spp. (e.g., C. diphtheriae), Ehrlichia spp. (E. canis, E. chaffeensis, etc.), Enterococcus spp. (E. faecalis, E. faecium), Escherichia spp. (e.g., E. coli (e.g., adherent-invasive E. coli, enterotoxigenic E. coli, enteropathogenic E. coli, etc.), Francisella spp. (e.g., F. tularensis), Haemophilus spp. (e.g., H. influenzae), Helicobacter spp. (e.g., H. pylori), Klebsiella spp. (e.g., K. pneumoniae), Legionella spp. (e.g., L. pneumophila), Leptospira spp., Listeria spp. (e.g., L. monocytogenes), Mycobacterium spp. (e.g., M. leprae, M. tuberculosis, etc.), Mycoplasma spp. (e.g., M. pneumoniae), Neisseria spp. (e.g., N. gonorrhoeae, N. meningitidis), Nocardia spp. (e.g., N. asteroids), Pseudomonas spp. (e.g., P. aeruginosa), Rickettsia spp. (e.g, R. rickettsii), Salmonella spp. (e.g., S. enterica, S. typhi, S. typhimurium, etc.), Shigella spp. (e.g., S. sonnei, S. dysenteriae), Staphylococcus spp. (S. aureus, S. epidermidis, S. saprophyticus, etc.), Streptococcus spp. (e.g., S. agalactiae, S. pneumoniae, S. pyogenes, S. viridans, etc.), Treponema spp. (e.g., T. pallidum), Vibrio spp. (e.g., V. cholerae), and Yersinia spp. (e.g., Y. pestis).

In some embodiments, a bacterial cell is a commensal bacterium. In some embodiments, a bacterial cell is a cell type found in human microbiota (e.g., bacteria of the phyla Firmicutes, Bacteroidetes, Proteobacteria, Verrumicrobia, Actinobacteria, Fusobacteria, or Cyanobacteria). In some embodiments, the microbiota is gastrointestinal microbiota, mucosal microbiota, skin microbiota, microbiota of the respiratory system, microbiota of the ear, nose, and throat, oral microbiota, or microbiota of the urinary tract. In some embodiments, the bacterial cell is of the genus Actinomyces, Akkermansia, Alistipes, Anaerofilum, Anaerostipes, Bacteroides, Barnesiella, Bifidobacterium, Blautia, Catabacter, Clostridium, Coprobacillus, Enterobacter, Enterococcus, Erysipelotrichaceae, Escherichia, Eubacterium, Faecalibacterium, Flavinofractor, Flavobacterium, Fusobacterium, Gordonibacter, Haemophilus, Holdemania, Hungatella, Lachnospiracea, Lactobacillus, Parabacteroides, Phascolarctobacterium, Prevotella, Pseudomonas, Robinsoniella, Romboutsia, Roseburia, Ruminococcus, Salmonella, Shigella, or Terrisporobacter, etc.

### Target Genes

The EVs (e.g., exosomes, microvesicles, etc.) of the disclosure may be used to target any suitable prokaryotic gene. In some embodiments, one or more prokaryotic genes (e.g., genes endogenous to prokaryotes) are targeted. In some embodiments, two or more prokaryotic genes (e.g., 2, 3, 4, 5, 6, 7, 8, 9, or 10 or more) are targeted. In certain embodiments, a single prokaryotic gene is targeted. In some embodiments, one or more genes in an operon are targeted. In some embodiments, an entire operon is targeted. A target gene may be located chromosomally or episomally.

In some embodiments, a target gene is an essential gene. An “essential gene” is a gene that is indispensable for the prokaryote to grow and/or divide. In some embodiments, an essential gene is associated with metabolism (e.g., catabolism, biosynthesis, etc.), cell division, DNA replication, membrane synthesis, etc. A number of essential genes have been characterized in the art (see, e.g., Goodall et al., mBio 9(1):e02096-17 (2018)).

In some embodiments, a target gene is associated with pathogenesis or modulation of host physiology. In some embodiments, a target encodes a gene product associated with antibiotic resistance, virulence, biofilm formation, stress response, protein export, bacterial secretion, amino acid biosynthesis, metabolic pathways, flagellar assembly, carbon fixation, adhesion, or iron acquisition. In some embodiments, a target gene encodes an enzyme, a toxin, an adhesin, a receptor, a membrane protein, a structural protein, or a secreted protein.

In some embodiments, the target gene is an antibiotic resistance gene. In some embodiments, an antibiotic resistance gene confers resistance to traditional small molecule antimicrobials. Examples of genes that confer aminoglycoside resistance include, without limitation, rpsL, rrnA, rrnB, aph, aac and aad variants and other genes that encode aminoglycoside-modifying enzymes. Examples of genes that confer beta-lactam resistance include, without limitation, genes that encode beta-lactamase (bla) (e.g., TEM, SHV, CTX-M, OXA, AmpC, IMP, VIM, KPC, NDM-1, family beta-lactamases) and mecA. Examples of genes that confer daptomycin resistance include, without limitation, mprF, yycFG, rpoB and rpoC. Examples of genes that confer macrolide-lincosamide-streptogramin B resistance include, without limitation, ermA, ermB and ermC. Examples of genes that confer quinolone resistance include, without limitation, qnrA, qnrS, qnrB, qnrC, qnrD, gyrA and parC. Examples of genes that confer trimethoprim/sulfonamide resistance include, without limitation, the dihydrofolate reductase (DHFR) and dihydropteroate synthase (DHPS) genes. Examples of genes that confer vancomycin resistance include, without limitation, vanA (e.g., vanRS and vanHAX), vanB and vanC operons. Examples of genes that encode multi-drug efflux pumps such as but not limited to, acrAB, mexAB, mexXY, mexCD, mefA, msrA and tetL. Antibiotic resistance genes are also described in the Comprehensive Antibiotic Resistance Database (CARD card.mcmaster.ca/).

In some embodiments, a target gene encodes a virulence factor. A virulence factor can be any substance produced by a pathogen that alter host-pathogen interaction by increasing the degree of damage done to the host. Virulence factors are used by pathogens in many ways, including, for example, in cell adhesion or colonization of a niche in the host, to evade the host's immune response, to facilitate entry to and egress from host cells, to obtain nutrition from the host, or to inhibit other physiological processes in the host. Virulence factors can include enzymes, endotoxins, adhesion factors, motility factors, factors involved in complement evasion, and factors that promote biofilm formation. Examples of genes that encode virulence factors include, without limitation, cytolethal distending toxins (CDT) toxins, such as cdtB), stx1 and stx2 (encode Shiga-like toxins), espA (responsible for induction of enterocyte effacement (LEE) A/E lesions), fimA (fimbriae major subunit), csgD (curli regulator) and csgA in E. coli; stx1 and stx2 in S. dysenteriae; yscF (plasmid-borne (pCD1) T3SS external needle subunit) in Y. pestis; fs1A in F. tularensis; pag (Anthrax toxin, cell-binding protective antigen) in B. anthracis; ctxA and ctxB (cholera toxin), tcpA (toxin co-regulated pilus), and toxT (master virulence regulator) in V. cholerae; genes that encode for the production of siderophore pyoverdine (e.g., sigma factor pvdS, biosynthetic genes pvdL, pvdl, pvdJ, pvdH, pvdA, pvdF, pvdQ, pvdN, pvdM, pvdO, pvdP, transporter genes pvdE, pvdR, pvdT and opmQ), genes that encode for the production of siderophore pyochelin (e.g., pchD, pchC, pchB, pchA, pchE, pchF and pchG, and genes that encode for toxins (e.g., exoU, exoS and exoT) in P. aeruginosa; fimA (adherence, type I fimbriae major subunit), and cps (capsular polysaccharide) in K. pneumoniae; ptk (capsule polymerization) and epsA (assembly) in A. baumannii; hilA (invasion, SPI-1 regulator), ssrB (SPI-2 regulator), and those associated with bile tolerance, including efflux pump genes acrA, acrB and tolC in S. Typhi, etc.

In some embodiments, a target gene encodes a toxin. A toxin may be an exotoxin, an endotoxin, or a genotoxin. Exotoxins are secreted while endotoxins remain a part of the prokaryote (e.g., bacteria). Examples of toxins include, without limitation, botulinum neurotoxins, tetanus toxin, Staphylococcus toxins, diphtheria toxin, anthrax toxin, alpha toxin, pertussis toxin, shiga toxin, heat-stable enterotoxin (E. coli ST), colibactin, B. fragilis toxin, cytolethal distending toxin (e.g., cdtB), C. difficile toxin, or any toxin described in Henkel et al., (Toxins from Bacteria in EXS. 2010; 100: 1-29).

### Methods of Use

The present disclosure provides a versatile technology for the genetic manipulation of prokaryotic cells. The compositions and methods of the disclosure may be used to deliver inhibitory nucleic acids targeting any desired prokaryotic gene. The delivery may be in vitro, or in vivo within a subject (e.g., to a prokaryote located in a subject or in an organ or tissue of the subject). The compositions and methods of disclosure may be used to regulate the expression of the desired prokaryotic gene. Without wishing to be bound by theory, an EV of the disclosure can interact with a target prokaryotic cell via membrane fusion and deliver the inhibitory nucleic acids to the cytoplasm of the target cell.

In one aspect, the disclosure provides a method of delivering one or more inhibitory nucleic acids (e.g., 1, 2, 3, 4, 5, 6, 7, 8, 9, or 10 or more) to a prokaryotic cell, comprising contacting the prokaryotic cell with an EV comprising the one or more inhibitory nucleic acids, wherein the one or more inhibitory nucleic acids target one or more genes in the prokaryotic cell, and wherein the EV is derived from a mammalian cell. In some embodiments, the EV additionally comprises one or more components of the endogenous mammalian machinery required for the processing and/or activity of the inhibitory nucleic acids. For example, an EV may further comprise one or more components of an RNA induced silencing complex (RISC) (e.g., protein kinase RNA activator (PACT), transactivation response RNA binding protein (TRBP), Dicer, Argonaute, Drosha, Pasha, etc.). The EVs of the disclosure may be used to deliver inhibitory nucleic acids to target any suitable prokaryotic gene in any suitable prokaryotic cell. In some embodiments, an EV is contacted with a prokaryotic cell at a ratio of 10 EVs to 1 CFU, 50 EVs to 1 CFU, 100 EVs to 1 CFU, 200 EVs to 1 CFU, 500 EVs to 1 CFU, or 1000 EVs to 1 CFU, etc. In some embodiments, an EV is contacted with a prokaryotic cell at a ratio of 100 EVs to 1 CFU.

In one aspect, the disclosure provides a method of regulating the expression of one or more target genes (e.g., 1, 2, 3, 4, 5, 6, 7, 8, 9, or 10 or more) in a prokaryotic cell, comprising contacting the prokaryotic cell with an EV comprising one or more inhibitory nucleic acids (e.g., 1, 2, 3, 4, 5, 6, 7, 8, 9, or 10 or more) that target one or more target, wherein the EV is derived from a mammalian cell. In some embodiments, the EV additionally comprises one or more components of the endogenous mammalian machinery required for the processing and/or activity of the inhibitory nucleic acids. For example, an EV may further comprise one or more components of an RNA induced silencing complex (RISC) (e.g., protein kinase RNA activator (PACT), transactivation response RNA binding protein (TRBP), Dicer, Argonaute, Drosha, Pasha, etc.). Following recognition of its target in the prokaryotic cell by the inhibitory nucleic acid, gene expression may be silenced or repressed (e.g., by transcriptional gene silencing, posttranscriptional gene silencing, RNAi, etc.)). Reduction in gene expression can be measured by any number of methods including reporter methods such as for example luciferase reporter assay, PCR-based methods, Northern blot analysis, Branched DNA, western blot analysis, and other techniques. In some embodiments, a reduction in gene expression may be at least 10%, at least 20%, at least 30%, at least 40%, at least 50%, at least 60%, at least 70%, at least 80%, or at least 90% lower compared to wild-type gene expression or expression prior to contacting the prokaryotic cell with an EV. In some embodiments, a reduction in gene expression may be 10-25%, 25-50%, 50-75%, or 75-100% lower compared to wild-type gene expression or expression prior to contacting the prokaryotic cell with an EV. In some embodiments, gene expression may be completely abrogated. In some embodiments, an EV is contacted with a prokaryotic cell at a ratio of 10 EVs to 1 CFU, 50 EVs to 1 CFU, 100 EVs to 1 CFU, 200 EVs to 1 CFU, 500 EVs to 1 CFU, or 1000 EVs to 1 CFU, etc. In some embodiments, an EV is contacted with a prokaryotic cell at a ratio of 100 EVs to 1 CFU.

In one aspect, the disclosure provides a method of modifying the composition of the microbiota in a subject, comprising administering to the subject EVs comprising one or more inhibitory nucleic acids (e.g., 1, 2, 3, 4, 5, 6, 7, 8, 9, or 10 or more) that target one or more genes (e.g., 1, 2, 3, 4, 5, 6, 7, 8, 9, or 10 or more) in a prokaryotic cell, and wherein the EVs are derived from a mammalian cell. In some embodiments, the EVs additionally comprises one or more components of the endogenous mammalian machinery required for the processing and/or activity of the inhibitory nucleic acids. For example, the EVs may further comprise one or more components of an RNA induced silencing complex (RISC) (e.g., protein kinase RNA activator (PACT), transactivation response RNA binding protein (TRBP), Dicer, Argonaute, Drosha, Pasha, etc.). Following recognition of its target in the prokaryotic cell by the inhibitory nucleic acid, gene expression may be silenced or repressed (e.g., by transcriptional gene silencing, posttranscriptional gene silencing, RNAi, etc.)). In some embodiments, the microbiota is gastrointestinal microbiota, mucosal microbiota, skin microbiota, microbiota of the respiratory system, microbiota of the ear, nose, and throat, oral microbiota, or microbiota of the urinary tract. Modifying the composition of the microbiota may comprise increasing or decreasing the diversity of species in the microbiota (e.g., by targeting a particular prokaryotic species for killing). Host physiology may be influenced by modulating gene expression in the microbiota of the subject. The ability to modulate genes expressed in prokaryotic cells in organisms that are part of a host microbiome (e.g., human microbiome) may also enable functional studies of the microbiome.

The compositions and methods of the disclosure allow the silencing of disease-causing genes in prokaryotic cells. Thus, in one aspect, the disclosure provides a method of treating or preventing a disease in a subject in need thereof, comprising administering to the subject extracellular vesicles (EVs) comprising one or more inhibitory nucleic acids (e.g., 1, 2, 3, 4, 5, 6, 7, 8, 9, or 10 or more) that target one or more genes (e.g., 1, 2, 3, 4, 5, 6, 7, 8, 9, or 10 or more) in a prokaryotic cell, and wherein the EVs are derived from a mammalian cell. In some embodiments, the EVs additionally comprises one or more components of the endogenous mammalian machinery required for the processing and/or activity of the inhibitory nucleic acids. For example, the EVs may further comprise one or more components of an RNA induced silencing complex (RISC) (e.g., protein kinase RNA activator (PACT), transactivation response RNA binding protein (TRBP), Dicer, Argonaute, Drosha, Pasha, etc.). Following recognition of its target in the prokaryotic cell by the inhibitory nucleic acid, gene expression may be silenced or repressed (e.g., by transcriptional gene silencing, posttranscriptional gene silencing, RNAi, etc.)).

Any disease, disorder, or condition related to prokaryotic gene expression (e.g., where regulation of prokaryotic gene expression may be desired) may be treated or prevented by the compositions and methods of the disclosure.

In some embodiments, the disease to be treated or prevented is associated with dysregulation of the microbiota in the subject. The EV composition may comprise inhibitory nucleic acids targeting a gene associated with modulation of host physiology. In some embodiments, the disease is a metabolic disorder, a cardiovascular disease, cancer, an autoimmune disease, cancer, or an inflammatory disease. In some embodiments, the disease is metabolic syndrome, inflammatory bowel disease, hypertension, asthma, diabetes, celiac disease, obesity, non-alcoholic fatty liver disease, non-alcoholic steatohepatitis, or rheumatoid arthritis. In some embodiments, the disease is cancer. In some embodiments, the cancer is a blood cancer (e.g., lymphoma, leukemia, multiple myeloma, etc.), breast cancer, prostate cancer, cancer of the digestive system (e.g. esophageal cancer, stomach cancer, colorectal cancer), liver cancer, cervical cancer, ovarian or uterine cancer, pancreatic cancer, lung cancer, brain cancer (e.g., glioblastoma), skin cancer (e.g., melanoma), or sarcomas of muscle or nerve, etc. In some embodiments, the disease is colorectal cancer.

In some embodiments, the disease to be treated or prevented is an infectious disease (e.g., an infection caused by a pathogenic bacterium). The EV composition may comprise inhibitory nucleic acids targeting a gene associated with pathogenicity. Examples of bacterial infections include, without limitation, actinomycosis, anthrax, whooping cough, pneumonia, Lyme disease, relapsing fever, brucellosis, enteritis, Guillain-Barre syndrome, trachoma, conjunctivitis, urethritis, pelvic inflammatory disease, epididymitis, prostatitis, lymphogranuloma venereum, psittacosis, botulism, pseudomembranous colitis, cellulitis, gas gangrene, food poisoning, tetanus, diphtheria, Ehrlichiosis, bacterial endocarditis, urinary tract infections, biliary tract infections, diarrhea, meningitis, sepsis, tularemia, respiratory tract infections, septic arthritis, peptic ulcer, gastritis, Legionnaire's disease, Pontiac fever, leptospirosis, listeriosis, leprosy, tuberculosis, gonorrhea, ophthalmia neonatorum, corneal infection, osteomyelitis, malignant external otitis, nocardiosis, keratitis, Rocky mount spotted fever, typhoid fever, paratyphoid fever, shigellosis, skin infections, toxic shock syndrome, scarlet fever, rheumatic fever, erysipelas, puerperal fever, necrotizing fasciitis, poststreptococcal glomerulonephritis, dental cavities, abscesses of brain and liver, syphilis, cholera, bubonic plague, pneumonic plague, etc.

### Pharmaceutical Compositions

The present disclosure provides compositions comprising EVs derived from mammalian cells and comprising one or more inhibitory nucleic acids targeting one or more prokaryotic genes. A pharmaceutical composition may comprise a plurality of EVs and a pharmaceutically acceptable carrier. The pharmaceutical composition may be in any form suitable for administration to a subject.

A pharmaceutically acceptable carrier is a pharmaceutically acceptable material, composition or vehicle, such as a liquid or solid filler, diluent, excipient, solvent or encapsulating material, involved in carrying or transporting a prophylactically or therapeutically active agent. Each carrier must be “acceptable” in the sense of being compatible with the other ingredients of the formulation and not harmful to the subject. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other non-toxic compatible substances employed in pharmaceutical formulations. Pharmaceutically acceptable carriers are determined in part by the particular composition being administered, as well as by the particular method used to administer the composition. See, for example, Remington's Pharmaceutical Sciences, Mack Publishing Co., Easton, Pa. 21st ed. (2005), incorporated herein by reference.

The EVs can be administered by parenteral, topical, intravenous, oral, subcutaneous, intra-arterial, intradermal, transdermal, rectal, intracranial, intraperitoneal, intranasal, intratumoral, or intramuscular routes or as inhalants. In some embodiments, the pharmaceutical composition comprising EVs is administered intravenously, e.g. by injection. A suitable route of administration allows the composition or the agent to perform its intended function. Administration includes self-administration and the administration by another.

The EVs can optionally be administered in combination with other therapeutic agents that are at least partly effective in treating the disease for which the EVs are intended. In some embodiments, a pharmaceutical composition comprises one or more other therapeutic agents and the EVs described herein. In some embodiments, the EVs are co-administered with one or more other therapeutic agents that are separately formulated, wherein co-administration includes administration of the other therapeutic agent before, after or concurrently with the administration of the EVs.

The EVs of the disclosure can be administered once, or alternatively they may be administered in a plurality of administrations. If administered multiple times, the EVs may be administered via different routes.

An effective amount of EVs may be administered to a subject. The absolute amount of EVs being administered will depend upon a variety of factors, including the material selected for administration, whether the administration is in single or multiple doses, and individual patient parameters including age, physical condition, size, weight, and the stage of the disease. These factors are well known to those of ordinary skill in the art.

### Kits

In one aspect, the present disclosure provides kits for delivery of one or more inhibitory nucleic acids to prokaryotic cells. In some embodiments, a kit comprises a composition comprising an effective amount of EVs according to the disclosure. In some embodiments, the kit comprises a sterile container which comprises the composition. Such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. If desired, the composition is provided together with instructions for administering the composition to a subject in need thereof.

### EXAMPLES

In order that the invention described herein may be more fully understood, the following examples are set forth. The Examples described in this Application are offered to illustrate the methods, compositions, and systems provided herein and are not to be construed in any way as limiting their scope.

### Example 1: EV Delivery of Inhibitory Nucleic Acids to Bacterial Cells

As a proof of concept of delivery of inhibitory nucleic acids to prokaryotic cells using EVs, it was tested whether EV (e.g., exosomal) delivery of an inhibitory RNA targeting the gentamicin acetyl transferase gene, which confers resistance to gentamicin, to E. coli cells stably expressing the gentamicin acetyl transferase gene, modulates gentamicin resistance.

Four siRNA duplexes targeting four different regions of the gentamicin acetyl transferase coding sequence were designed and lipofectamine transfected into HCT116 colorectal cancer cells. Four siRNA duplexes without any specificity for the gentamicin acetyl transferase coding sequence were used as inactive controls. The cells were cultured in the presence of 10% EV-depleted FBS. Media was collected after two days of culture. To stress the EVs and increase the EV yield, media was replaced with serum-free media and cultured for 24h. Serum-containing and serum-free media were combined and EVs were isolated using differential ultracentrifugation.

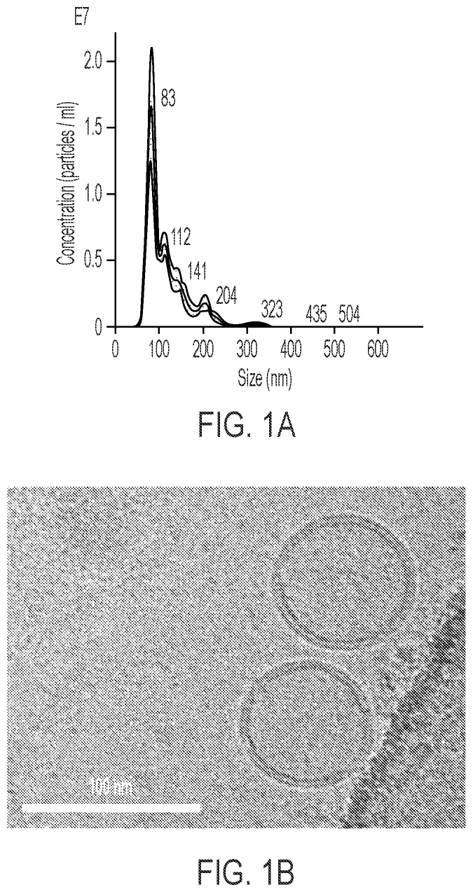

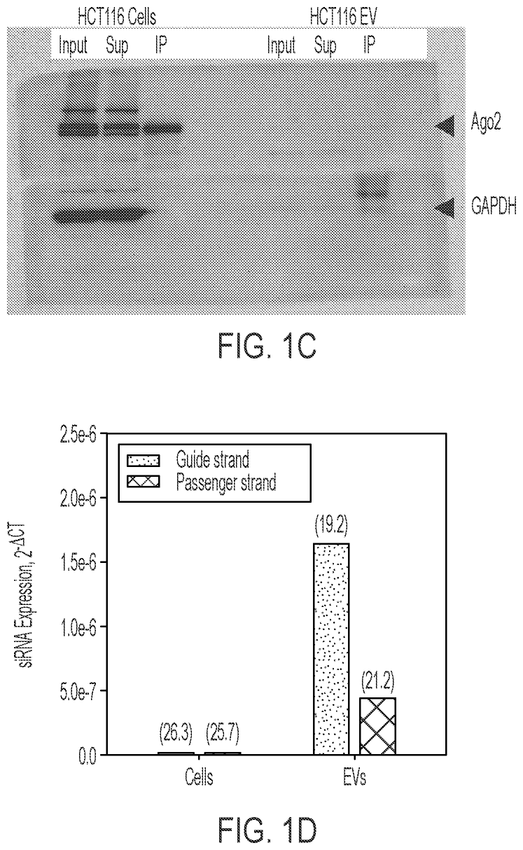

Characterization of EVs is shown in FIGS. 1A-1D. The particle size and concentration of the EVs were determined using Nanotracking Analysis and their morphology was determined using cryo transmission electron microscopy (FIGS. 1A-1B). Further, Ago2 and siRNA levels in the EVs were characterized. HCT116 cells were electroporated with siRNA to the antibiotic resistance cassette and collected using differential electroporation. Protein isolated from the HCT116 cells (left) or HCT116 EVs (right) were immunoprecipitated using an antibody to Ago2. The immunoprecipitated (IP) protein, input or supernatant was resolved on a denaturing polyacrylamide gel, transferred to nitrocellulose and probed using antibodies for Ago2 (upper) or GAPDH (lower) (FIG. 1C). RNA was isolated from HCT116 cells or HCT116 EVs from HCT116 cells lipofected with siRNA to the antibiotic resistance cassette. cDNA synthesized using looped primers to the guide or passenger strands of the siRNA were quantified by qPCR (FIG. 1D).

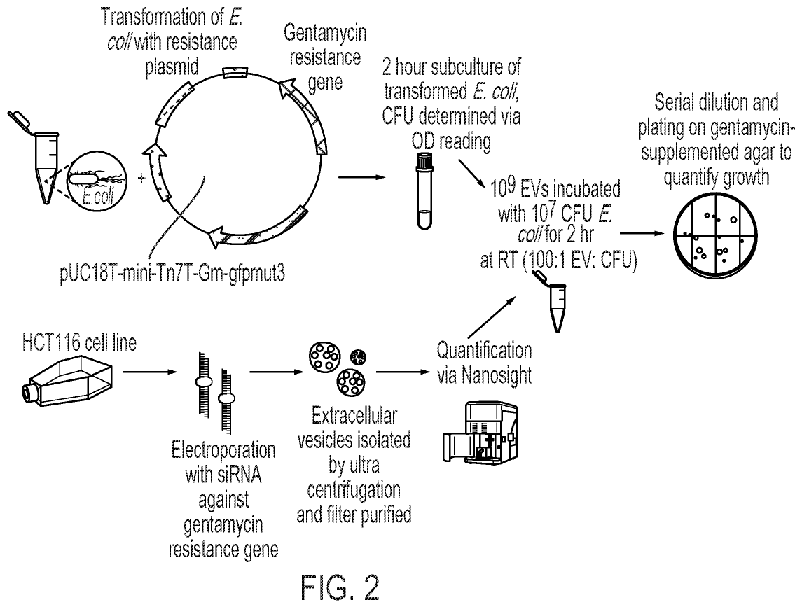

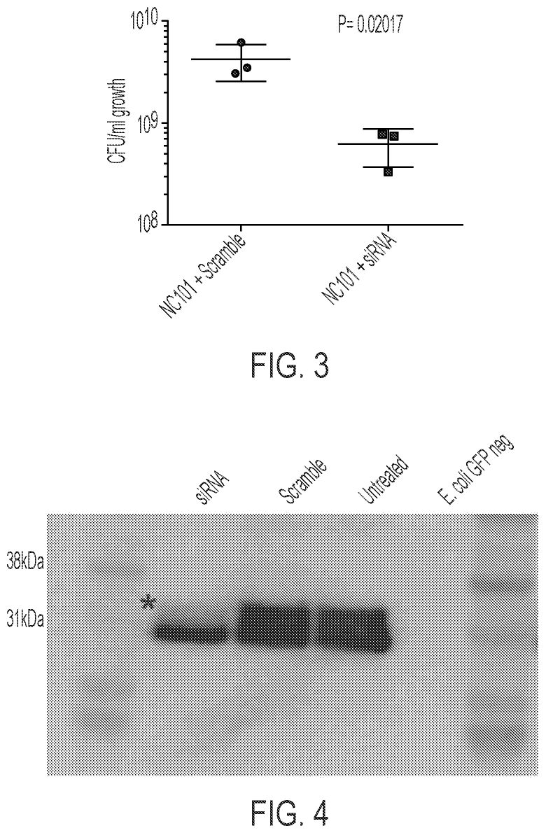

E. coli cells were transformed with the gfpmut3 plasmid containing the gentamicin acetyl transferase gene, to render this bacterium resistant to gentamicin exposure. EVs (e.g., exosomes) from siRNA-transfected HCT116 cells were isolated, purified, and incubated with E. coli cells (FIG. 2). The E. coli cells were exposed to EVs loaded with either siRNA targeting the gentamicin acetyl transferase gene or control scrambled siRNA for 2 hours at a ratio of 100 EVs to 1 CFU of E. coli. The treated bacteria were then plated at dilutions between 10⁻⁶ to 10⁻⁸ in triplicate on 20 μg/mL gentamicin-supplemented LB plates overnight. The number of colonies per dilution were counted the next day and the number of CFU/mL of bacterial growth was calculated. As shown in FIG. 3, E. coli growth (cfu count) was inhibited by 1 log (10-fold) in the presence of functional siRNA compared to inactive siRNA.

In a separate experiment, whether EV (e.g., exosomal) delivery of siRNA against GFP would block GFP expression in E. coli cells stably expressing this protein was tested. E. coli cells stably expressing the gfpmut3 plasmid were exposed to EVs loaded with either siRNA targeting the gfpmut3 gene or control scrambled siRNA for 2 hours at a ratio of 100 EVs to 1 CFU of E. coli. The treated bacteria were then lysed, and 15 μg of protein from each lysate was analyzed by Western blot assay using anti-GFP primary antibody from GeneTex. As shown in FIG. 4, GFP expression was inhibited in E. coli exposed to GFP-specific siRNA but not non-specific siRNA.

These data demonstrate that exosomes can be used to specifically deliver functional, inhibitory nucleic acids and target microbial genes.

### Example 2: Human Colon Mucosal Biofilms and Murine Host Communicate Via Altered mRNA and MicroRNA Expression During Cancer

Example 2 relates to Tomkovich et al., Host-Microbe Biology, 5(1):e00451-19 (2020), the contents of which are incorporated by reference herein. Disrupted interactions between host and intestinal bacteria are implicated in colorectal cancer (CRC) development. However, activities derived from these bacteria and their interplay with the host are unclear. This interplay was examined by performing mouse and microbiota RNA sequencing on colon tissues and 16S and small RNA sequencing on stools from germfree (GF) and gnotobiotic Apc^{MinΔ850/+};Il10^{-/-} mice associated with microbes from biofilm-positive human CRC tumor (BF+T) and biofilm-negative healthy (BF-bx) tissues. The bacteria in BF+T mice differentially expressed (DE)>2,900 genes, including genes related to bacterial secretion, virulence, and biofilms but affected only 62 host genes. Small RNA sequencing of stools from these cohorts revealed eight significant DE host microRNAs (miRNAs) based on biofilm status and several miRNAs that correlated with bacterial taxon abundances. Additionally, computational predictions indicate that some miRNAs preferentially target bacterial genes while others primarily target mouse genes. 16S rRNA sequencing of mice that were reassociated with mucosa-associated communities from the initial association revealed a set of 13 bacterial genera associated with cancer that were maintained regardless of whether the reassociation inoculums were initially obtained from murine proximal or distal colon tissues. Without wishing to be bound by theory, the data indicate that complex interactions within bacterial communities affect host-derived miRNA, bacterial composition, and CRC development.

Bacteria and bacterial biofilms have been implicated in colorectal cancer (CRC), but it is still unclear what genes these microbial communities express and how they influence the host. MicroRNAs regulate host gene expression and have been explored as potential biomarkers for CRC. An emerging area of research is the ability of microRNAs to impact growth and gene expression of members of the intestinal microbiota. This study examined the bacteria and bacterial transcriptome associated with microbes derived from biofilm-positive human cancers that promoted tumorigenesis in a murine model of CRC. The murine response to different microbial communities (derived from CRC patients or healthy people) was evaluated through RNA and microRNA sequencing. A complex interplay between biofilm-associated bacteria and the host during CRC in mice was identified. These findings may lead to the development of new biomarkers and therapeutics for identifying and treating biofilm-associated CRCs.

Numerous 16S rRNA and shotgun metagenomic studies have demonstrated that colorectal cancer (CRC) patients have an altered intestinal microbiota compared to healthy controls. Colibactin-producing Escherichia coli, enterotoxigenic Bacteroides fragilis, and Fusobacterium nucleatum among others, are implicated in CRC pathogenesis due to their abilities to produce genotoxins and adhesins which promote proliferation and modulate immune responses in preclinical models. How these bacteria interact with the rest of the complex microbiota to influence CRC initiation and/or progression is still unclear. In fact, recent studies with F. nucleatum indicated these bacteria may be associated with later stages of disease and have less of an influence on CRC initiation. Testing the functional role of human CRC-associated bacterial communities in chemically induced mouse models of CRC have led to mixed results. One group demonstrated stool communities from either individual CRC or healthy patients promoted polyp formation depending on the composition of the microbiota that established in mice. Another recent report revealed an increased tumorigenic phenotype in mice that received stools pooled from multiple CRC patients compared to stools from controls.

The lack of a consensus carcinogenic CRC-associated microbiota from patient stools indicates that other factors, including how the bacteria are organized/located or the genes they express, may be just as important to CRC pathogenesis. Polymicrobial bacterial biofilms, spanning>200 μm of epithelial surface were recently identified in ˜52% of human CRC patients and also found in ˜13% of the healthy patients who were screened. It was previously shown that human biofilm-forming bacterial communities from either CRC or healthy patients play a functional role in CRC development in multiple preclinical mouse models, emphasizing the contribution of bacterial organization to CRC.

CRC is an evolving disease, characterized by a series of molecular and microbial changes, that indicates a dynamic interplay between the host and intestinal microbiota as the disease progresses. MicroRNAs (miRNAs) have emerged as potential mediators of these host-microbe interactions with their ability to modulate both host and bacterial genes, which can result in shifts in microbiota composition. In turn, the microbiota is able to modulate host miRNA expression, with F. nucleatum targeting several miRNAs related to CRC pathogenesis. However, it is uncertain how human CRC-associated microbial communities as a whole impact fecal miRNA expression and whether host miRNAs affect bacterial composition/gene expression during CRC.

To examine the bacterial activities associated with biofilm-positive microbes from CRC patients, mouse and bacterial gene expression were examined from colon tissues and mouse small RNA sequencing from stools collected from biofilm-positive associated Apc^{MinΔ850/+};Il10^{-/-} mice. It was found that a number of bacterial virulence genes were increased in biofilm-positive communities and identified a conserved core group of transmissible biofilm-positive associated bacteria. Additionally, it was demonstrated that biofilm status and CRC development alter miRNA expression and specific miRNAs correlate with biofilm-positive associated taxa.

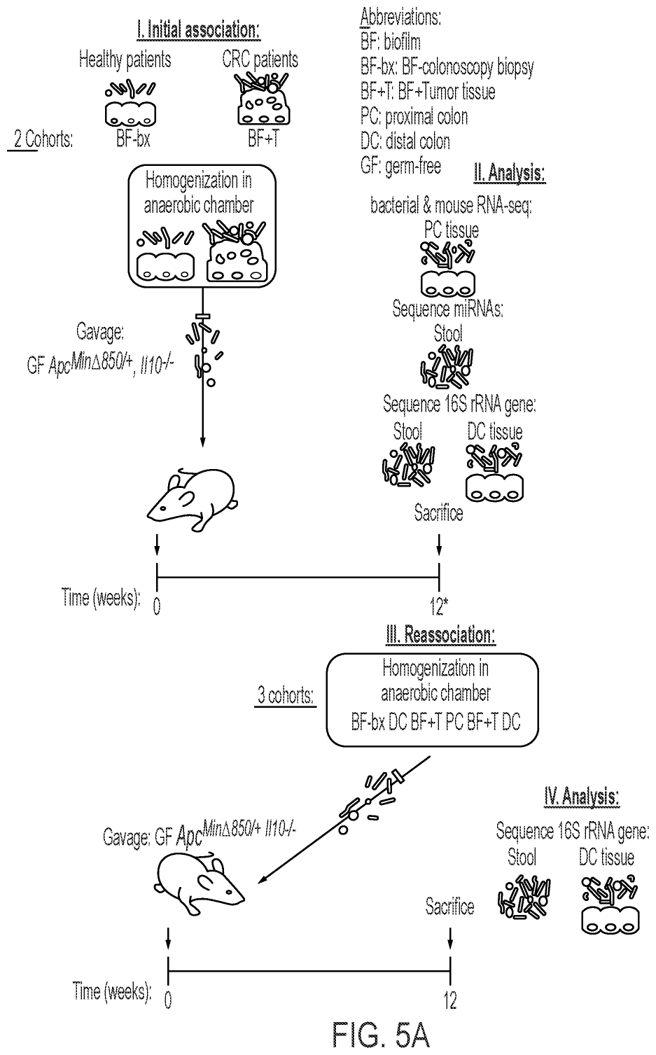

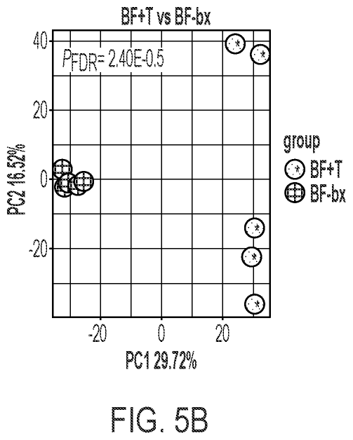

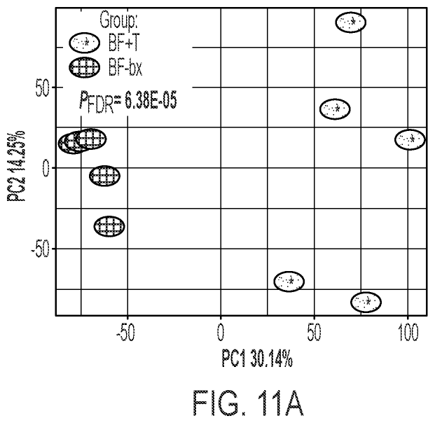

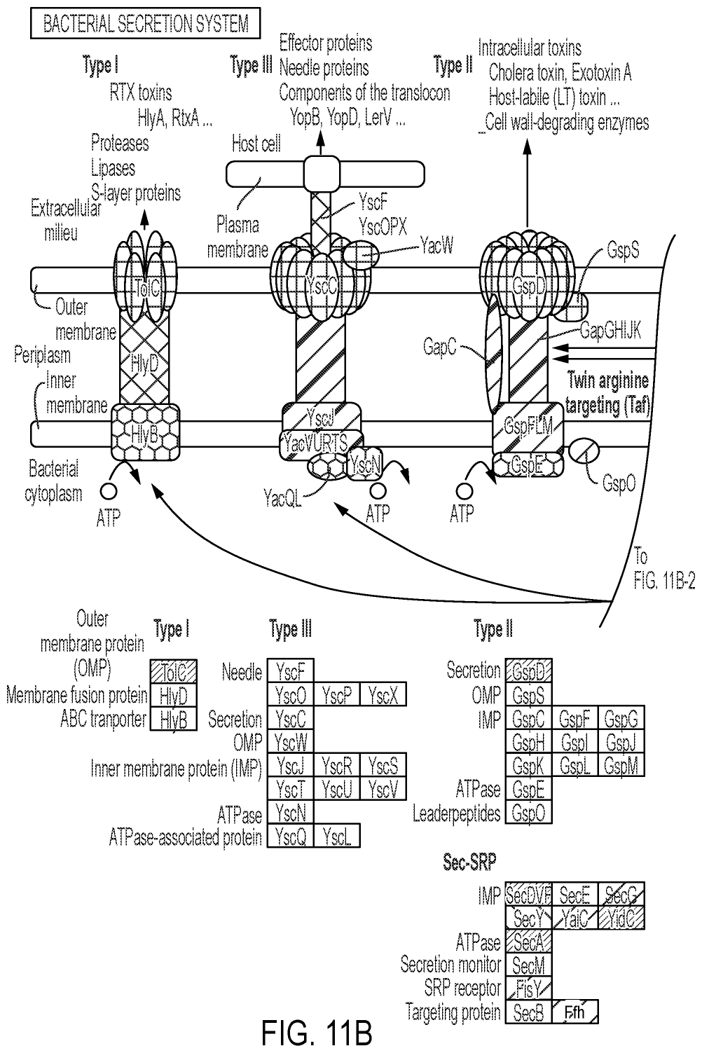

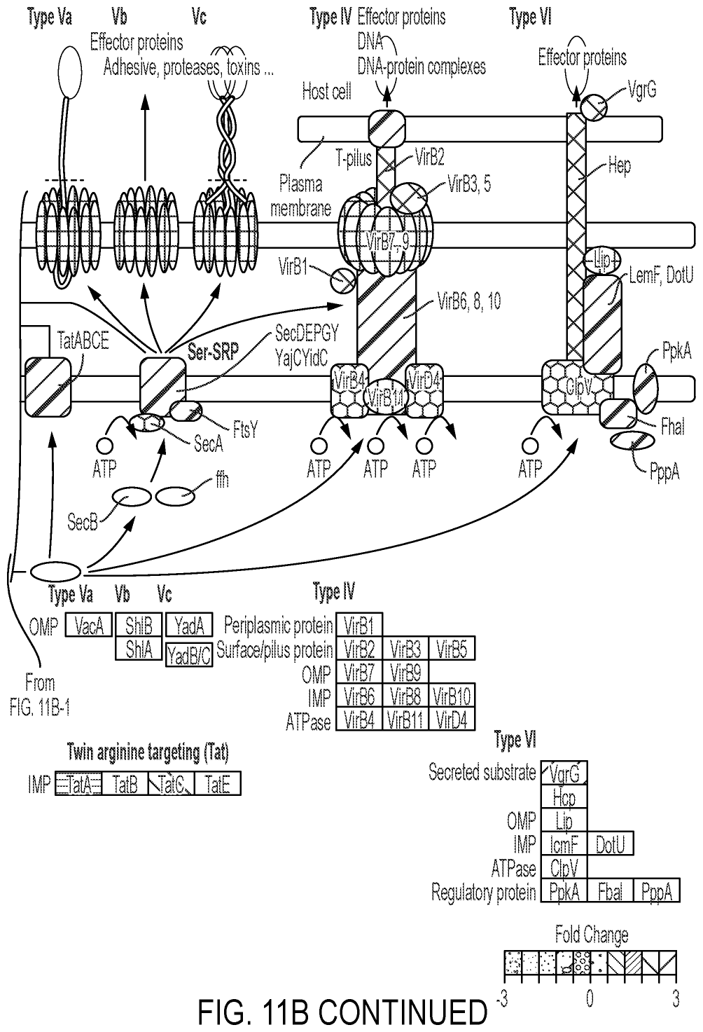

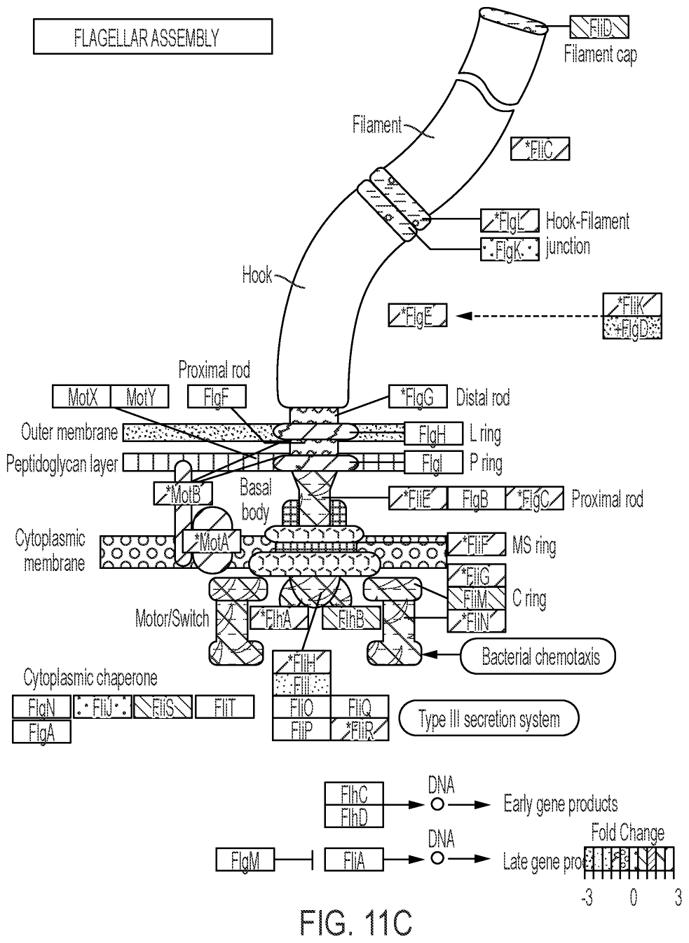

Bacterial activities associated with biofilm status. In order to elucidate microbial activities associated with biofilm-forming bacteria derived from human CRC patients that promote tumorigenesis in Apc^{MinΔ850/+};Il10^{-/-} mice, mouse and microbial gene expression from colon tissue snips was characterizing using RNA sequencing (see FIGS. 5A I and II for experimental design). Principal-component analysis (PCA) of microbial community gene expression detected by both the de novo assembly and aligning the microbial transcriptome sequencing (RNA-seq) reads to the human gut microbiome integrated gene catalog (IGC) showed separate clustering of biofilm-positive CRC tumor tissue (BF+T) associated Apc^{MinΔ850/+};Il10^{-/-} mice from biofilm-negative healthy patient tissue (BF-bx) associated mice (FIG. 5B and FIG. 11A). Bacterial metatranscriptomic analysis found 2,918 significant differentially expressed (DE) genes (false-discovery rate-adjusted P value [P_{FDR}]<0.05), the majority of which were increased in BF+T mice (2,739 increased genes and 179 decreased genes) compared to BF-bx mice. Pathways related to protein export, bacterial secretion systems, carbon fixation, flagellar assembly, and biosynthesis of amino acids were increased in BF+T mice compared to BF-bx mice (Table 1 and FIG. 11B-11C). Additional genes related to virulence and biofilm formation, including stress response, toxins, iron acquisition, mucin cleavage/transport, outer membrane polysaccharide importers, and adhesins were also significantly increased in BF+T mice. Increased toxin genes included Clostridium difficile toxins A and B, Clostridium perfringens Mu toxin, and E. coli colibactin (clbG and clbI). Weighted gene coexpression network analysis identified 34 hub genes from modules detected in BF+T mice and included outer membrane proteins involved in protein export and heat shock proteins involved in the stress response.

TABLE 1
Pathways enriched in BF + T samples using KEGG pathways
from Trinity assembly or HUMAnN analysis of RNA-seq reads

| | | |
|---|---|---|
| Method and pathway | P value | PFDR |

Trinity assembly

| | | |
|---|---|---|
| Carbon fixation pathways in prokaryotes | 1.06e−05 | 0.0006 |
| Protein export | 0.0002 | 0.0051 |
| Bacterial secretion system | 0.0002 | 0.0051 |
| Valine, leucine, and isoleucine biosynthesis | 0.0004 | 0.007 |
| 2-Oxocarboxylic acid metabolism | 0.001 | 0.0131 |

HUMAnN analysis

| | | |
|---|---|---|
| 2-Oxocarboxylic acid metabolism | 1.41e−05 | 0.0007 |
| Inositol phosphate metabolism | 1.42e−05 | 0.0007 |
| Biosynthesis of amino acids | 0.0004 | 0.0163 |
| Flagellar assembly | 0.001 | 0.0204 |
| Protein export | 0.0021 | 0.035 |

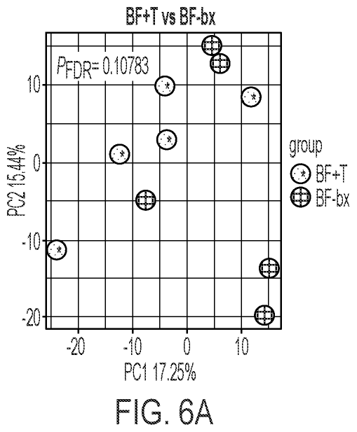

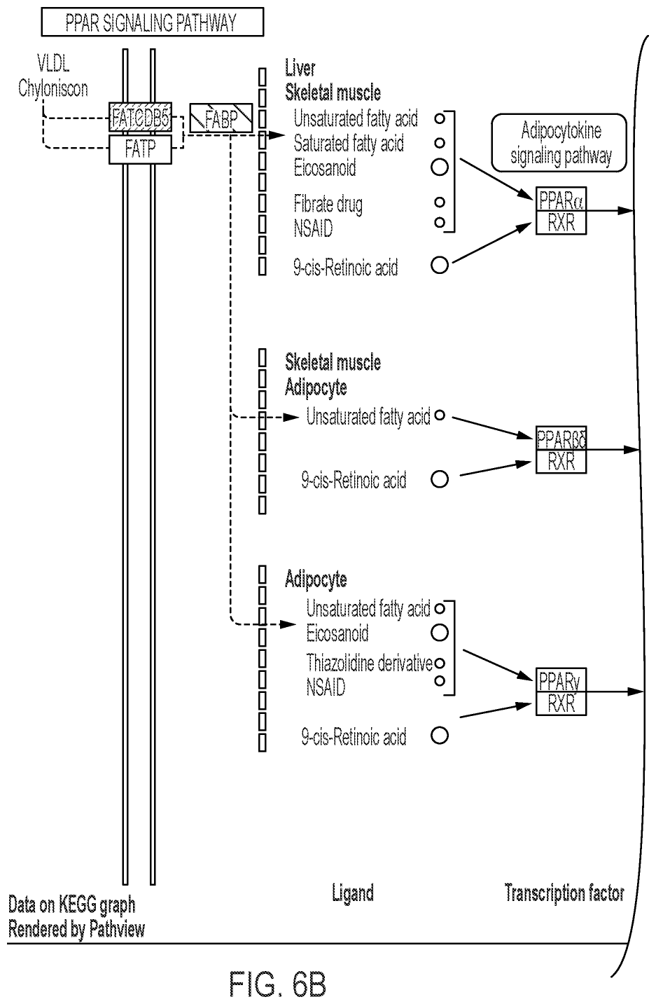

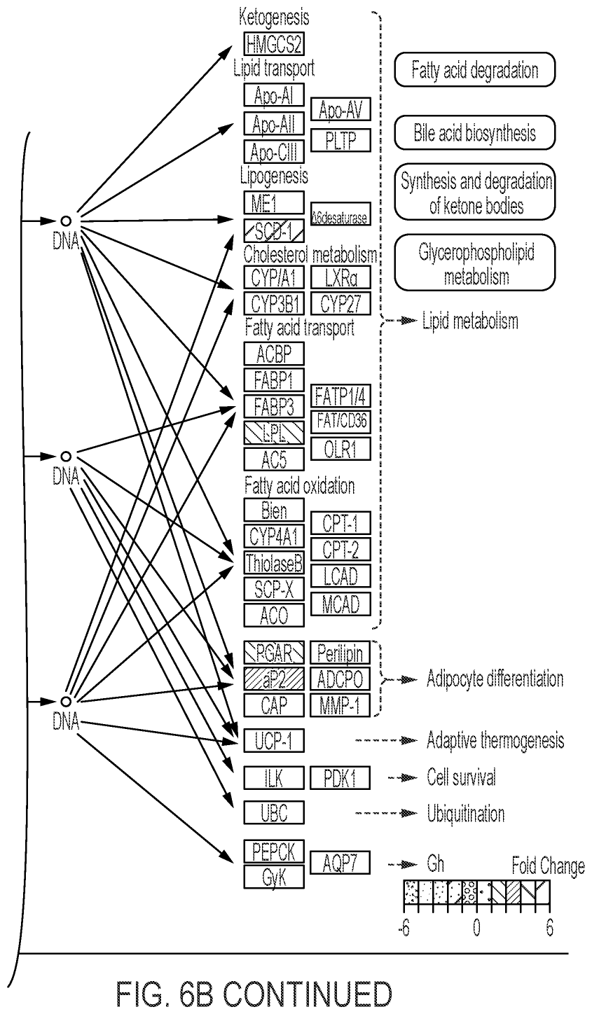

Despite the high number of microbial genes with increased expression in BF+T microbial communities, no separation by PCA analysis was found at the host gene expression level, and only 62 significant DE genes (FIG. 6A) between BF+T and BF-bx mice were detected. Instead, the host was more responsive to the microbiota in general as opposed to the type of microbiota, since the host transcriptomes of either BF+T- or BF-bx-associated mice clustered separately from germfree (GF) mice. There were >3,000 significant DE host genes (˜2,000 upregulated, ˜1,300 downregulated) in the BF+T and BF-bx groups compared to GF mice and pathway analysis revealed that the majority of upregulated genes in colonized mice belonged to immune-related pathways. Only the peroxisome proliferator-activated receptor (PPAR) signaling pathway was significantly upregulated (P=1.88e-05, PFDR=0.004) in BF+T mice compared to BF-bx mice. The majority of the significant DE genes were upregulated (56 versus 6 downregulated) in BF+T mice and included genes related to lipid metabolism, iron scavenging, and the extracellular matrix. Cross-referencing the list of upregulated genes with The Cancer Genome Atlas (TCGA) colorectal microarray data using Oncomine revealed 10 of the upregulated genes were also significantly overexpressed in colon adenocarcinomas compared to control tissues with SCD (stearoyl-coenzyme A [CoA] desaturase, PPAR pathway member), MMP10, and SLC22A3 increased more than 2-fold.

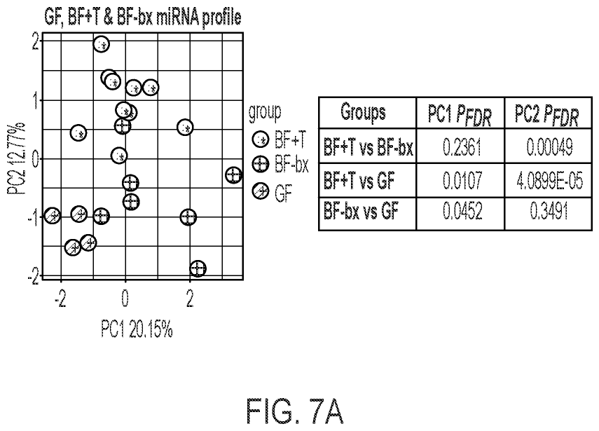

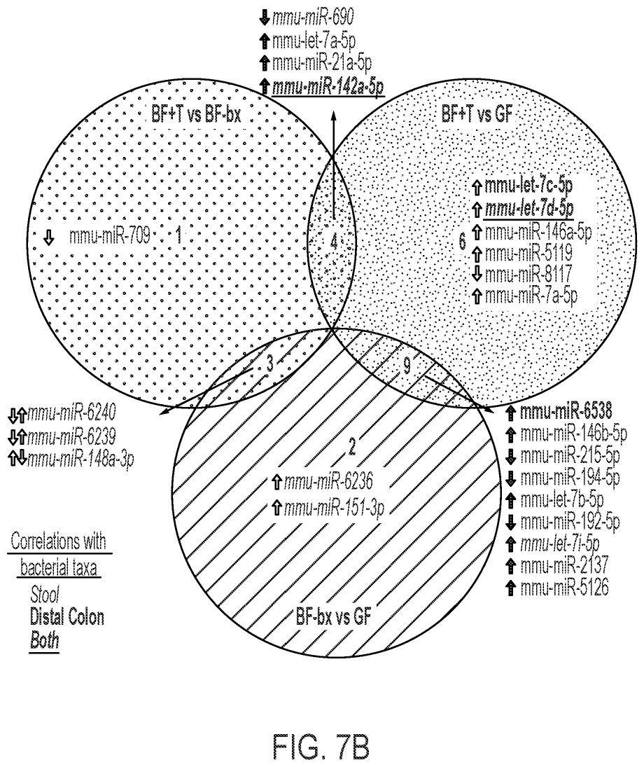

Biofilm status alters the fecal miRNA profile. Given the differential bacterial and host gene expression observed in BF+T mice that developed colon tumors and the potential of miRNAs to modulate interkingdom interactions, stool miRNA expression was next profiled. To examine the interplay between miRNAs and bacterial communities derived from human CRC or healthy patients, the fecal small RNAs from GF, BF-bx, and BF+T Apc^{MinΔ850/+}; Il10^{-/-} mice was sequenced. The PCA plot shows clear separation between GF mice and BF-bx or BF+T-associated mice, demonstrating that microbial colonization alters the fecal miRNA profile in Apc^{MinΔ850/+};Il10^{-/-} mice (FIG. 7A). Biofilm/cancer status of the initial human-derived microbes also modulates miRNA expression since PCA analysis demonstrates separation of BF+T and BF-bx miRNAs (FIG. 7A). Pair-wise comparisons between the three groups of mice revealed that 25 unique miRNAs were significantly DE (out of 142 total detected) (FIG. 7B). Next, the significantly different miRNAs in BF+T or BF-bx versus GF mice were compared and it was found that nine significant DE miRNAs overlapped (mmu-miR-6538, -146b-5p, -215-5p, -194-5p, -192-5p, -2137, and -5126 and mmu-let-7b-5p and mmu-let-7i-5p), indicating host miRNAs targeting the microbiota (FIG. 7B). Eight miRNAs were also identified (mmu-miR-709, -690, -21a-5p, -142a-5p, -6240, -6239, and -148a-3p and mmu-let-7a-5p) that were significantly DE according to biofilm status (BF+T versus BF-bx [FIG. 7B]). These data indicate that the microbiota modulates host miRNA expression. Together, both microbiota composition and disease status drive miRNA expression (BF+T versus BF-bx DE miRNAs).

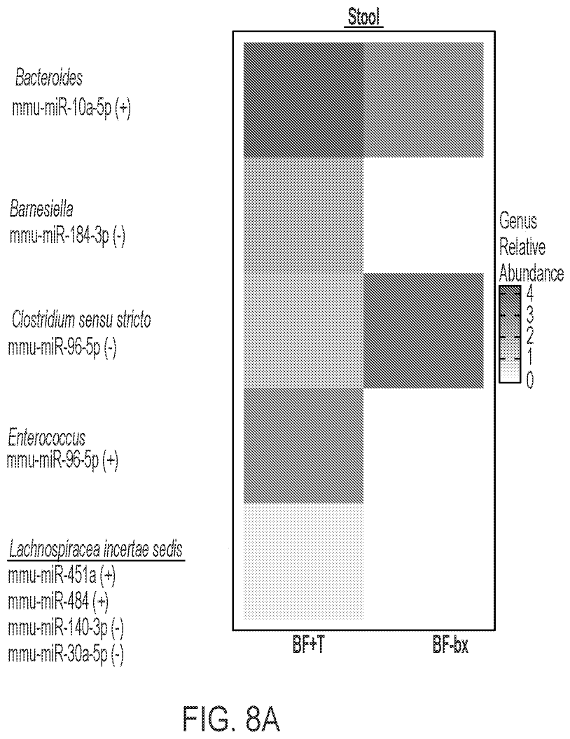

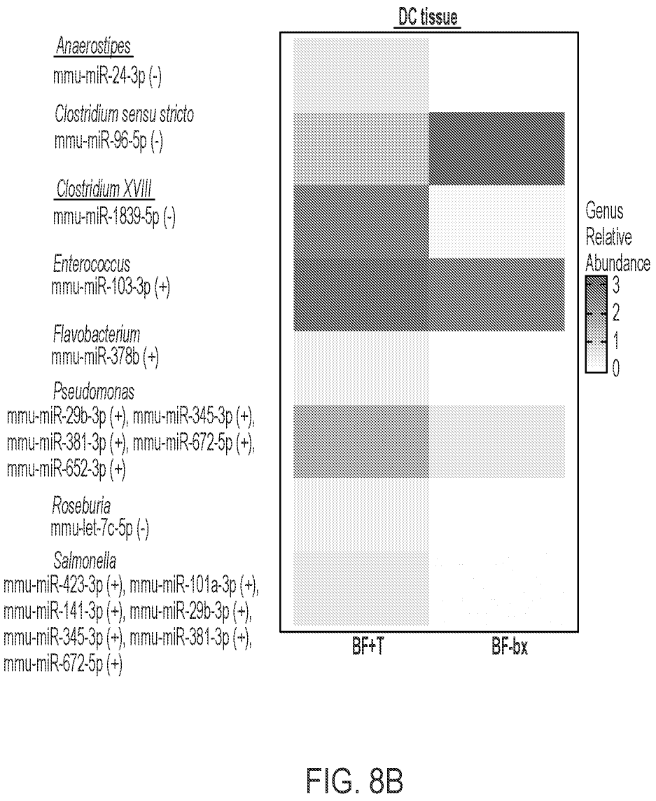

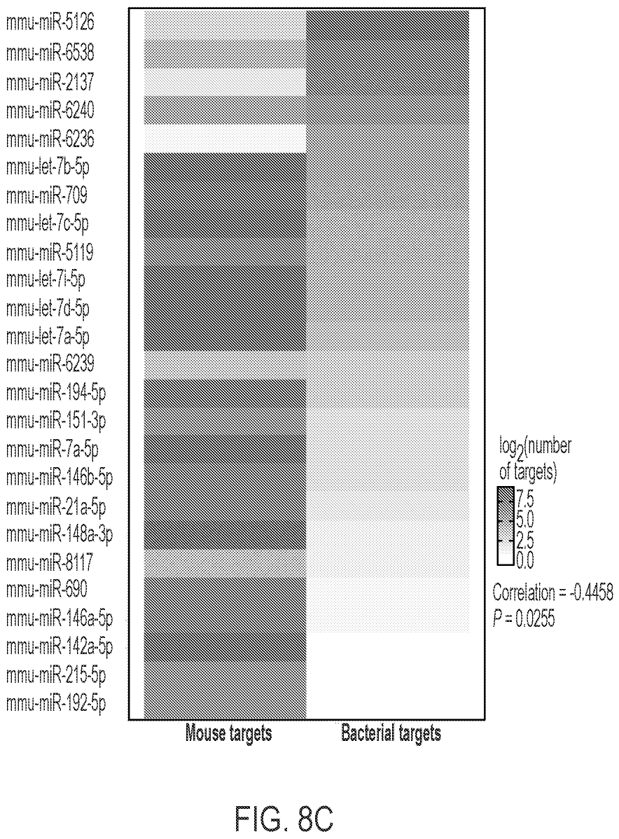

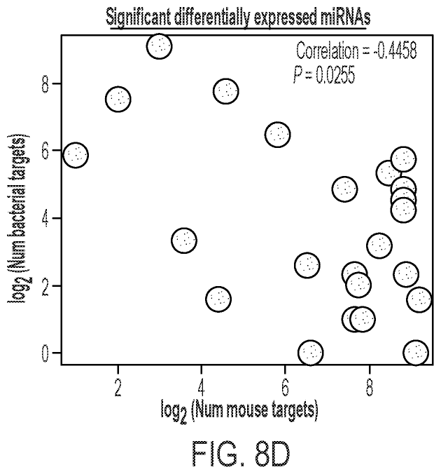

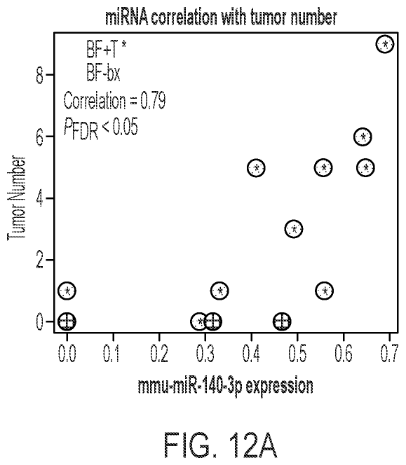

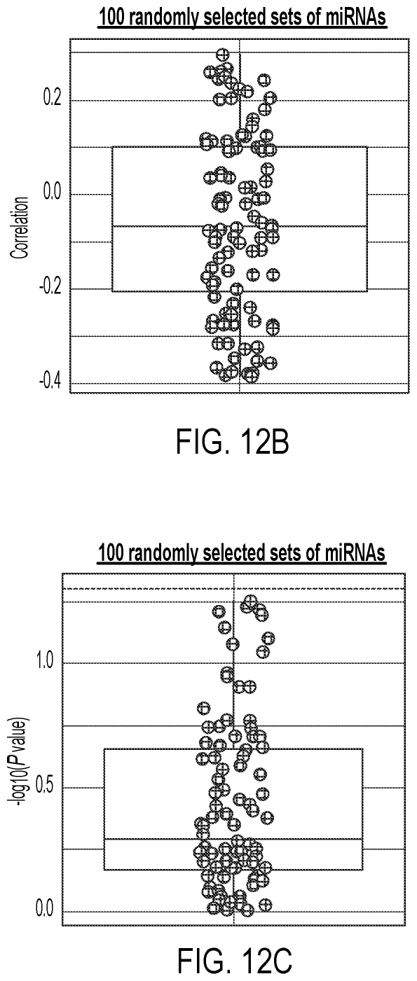

Fecal miRNAs correlate with specific bacterial taxa and target bacterial and mouse genes. The fecal miRNAs of human microbiota-associated mice were next examined for possible correlation with bacterial taxa previously identified in the mice. 11 miRNAs were identified that were significantly DE between GF, BF-bx, and BF+T mice and correlated with the relative abundances of bacterial taxa in the stool, distal colon tissue, or both compartments (FIG. 7B [colored green, orange, or underlined]). Additionally, miRNAs were identified that although not significantly DE between groups, correlated with five and eight bacterial genera in the stool and distal colon tissues, respectively (FIGS. 8A-8B). Five of these genera (Bacteroides, Lachnospiracea incertae sedis, Anaerostipes, Clostridium XVIII, and Roseburia) were significantly increased in BF+T mice (FIGS. 8A-8B). Furthermore, mmu-miR-140-3p correlated with Lachnospiraceae incertae sedis abundance and tumor number, both of which were increased in BF+T mice (FIG. 8A and FIG. 12A). Thus, CRC-associated microbial communities elicit a specific host miRNA profile and maintain a subset of miRNAs that correlate with specific microbial taxa.

Interestingly, computationally predicting the bacterial genes targeted by the significant miRNAs revealed several miRNAs (mmu-miR-2137, mmu-miR-5126, and mmu-miR-6538) that primarily target bacterial genes. In contrast, other miRNAs (mmu-let-7s, mmu-miR-21a-5p, mmu-miR-142a-5p, mmu-miR-148a-3p, mmu-miR-194-5p, mmu-miR-690, and mmu-miR-709) are predicted to primarily target mouse genes, and there is a significant negative correlation between the number of predicted bacterial versus mouse gene targets for the significant miRNAs were identified (FIGS. 8C-8D and FIGS. 12B-12C). Taken together, these predictions indicate that specific miRNAs have differential roles in mediating host-microbe interactions, with some mainly modulating host gene expression and others primarily impacting bacterial gene expression and/or abundance.

A core BF+T microbiota is transmissible. Carcinogenic properties are retained over time as microbial inoculums from homogenized mouse colon tissues collected from the initial BF+T but not BF-bx tissue-associated microbes promoted tumors in new cohorts of GF Apc^{MinΔ850/+};Il10^{-/-} mice (FIG. 5A III).

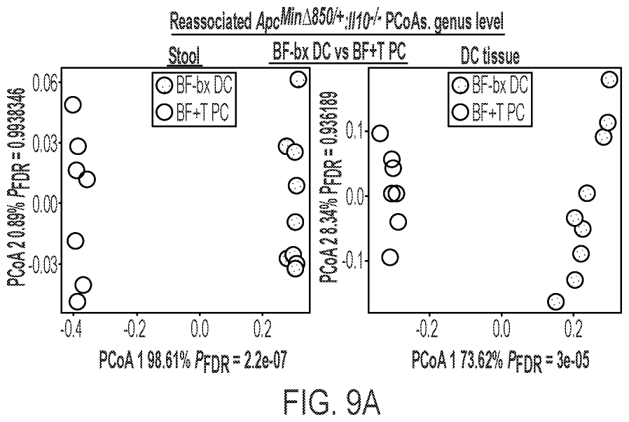

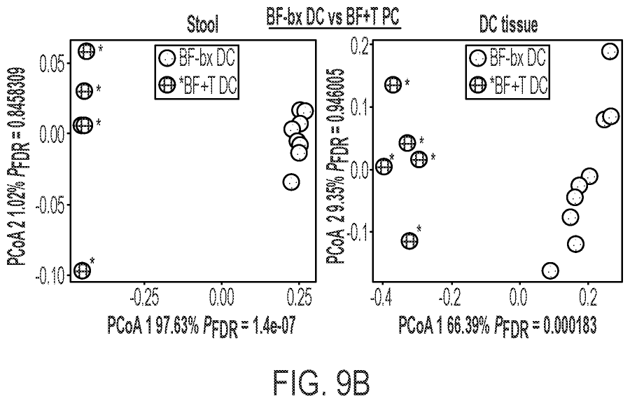

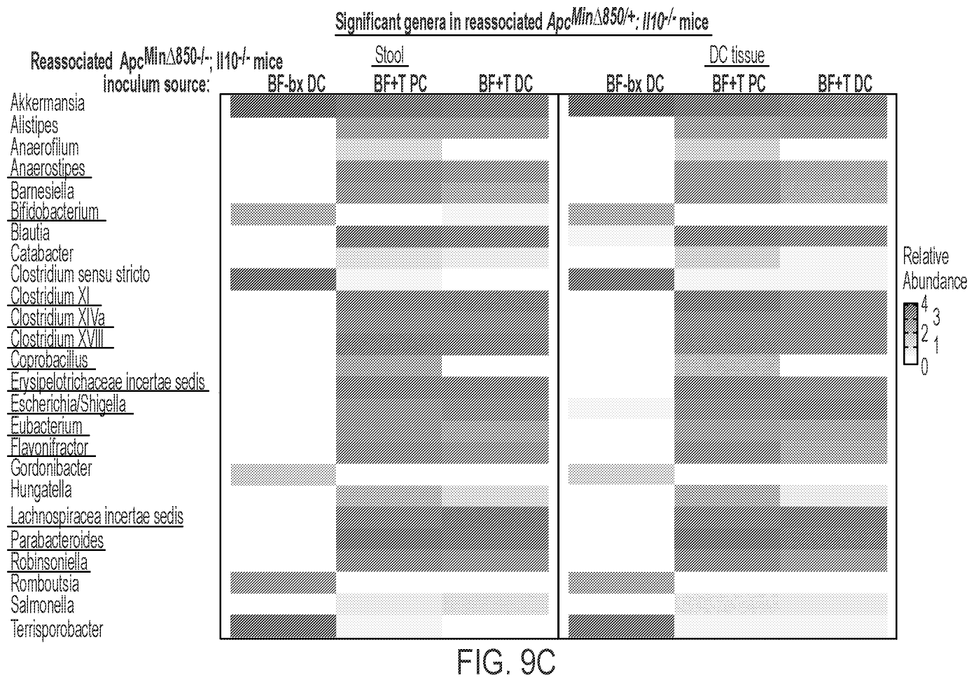

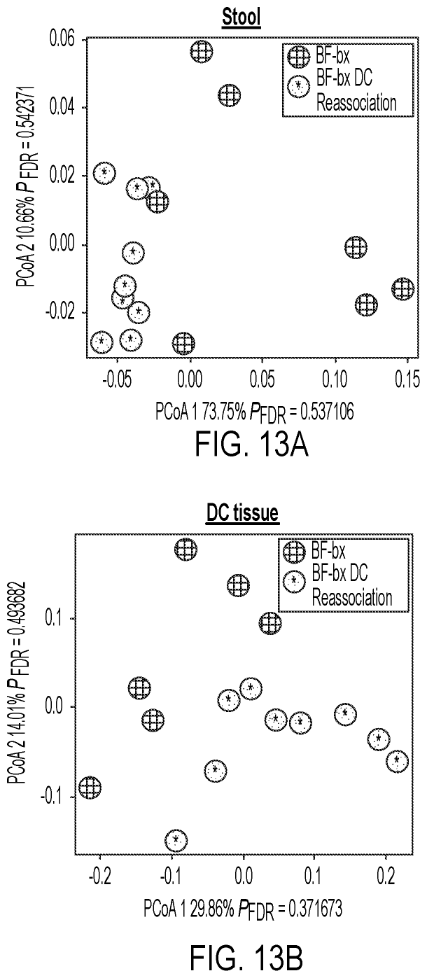

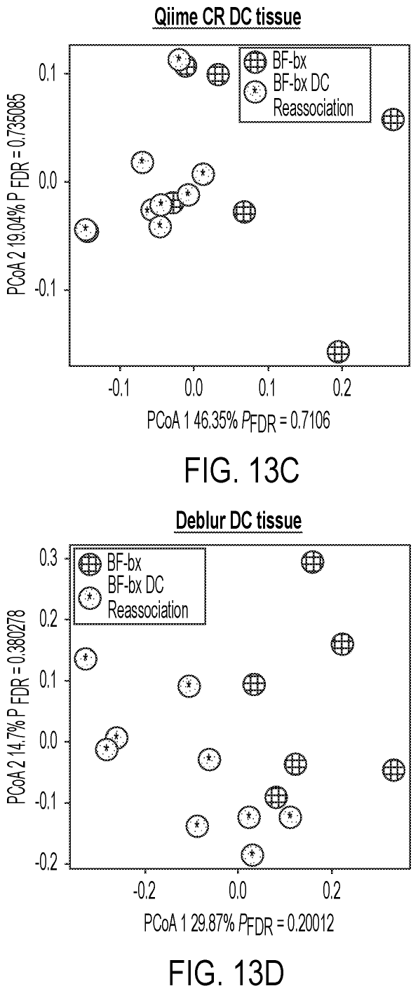

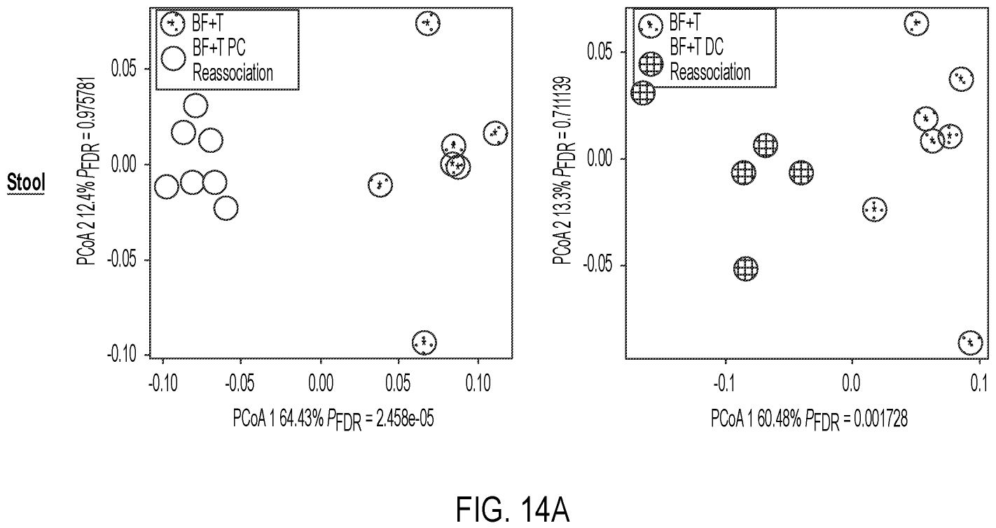

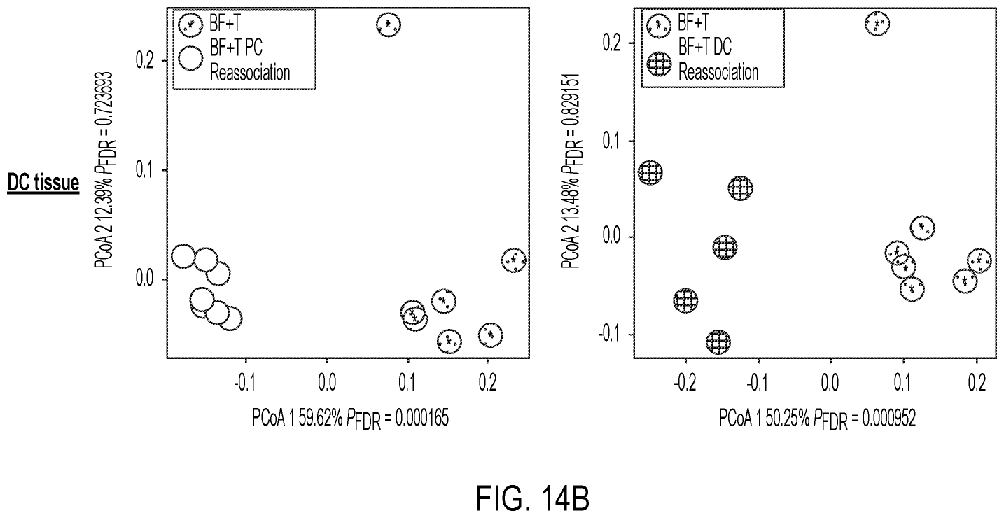

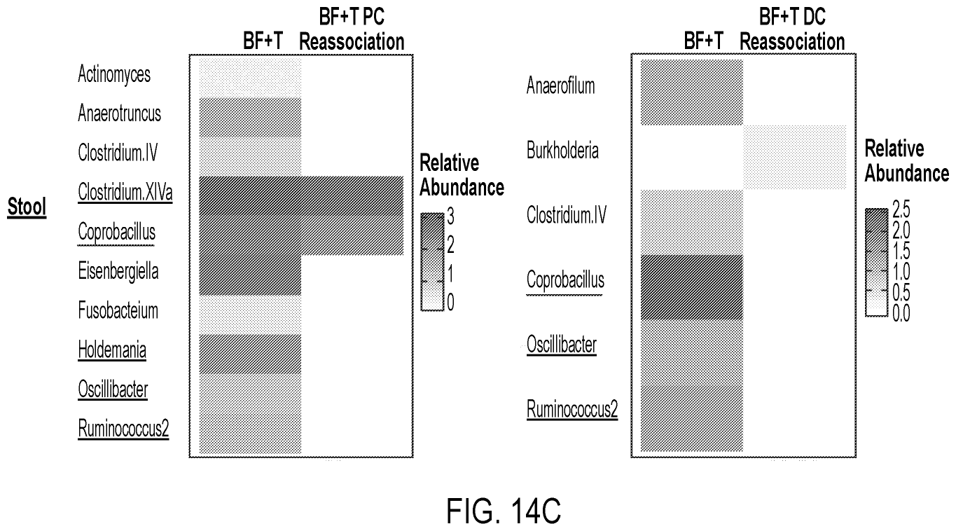

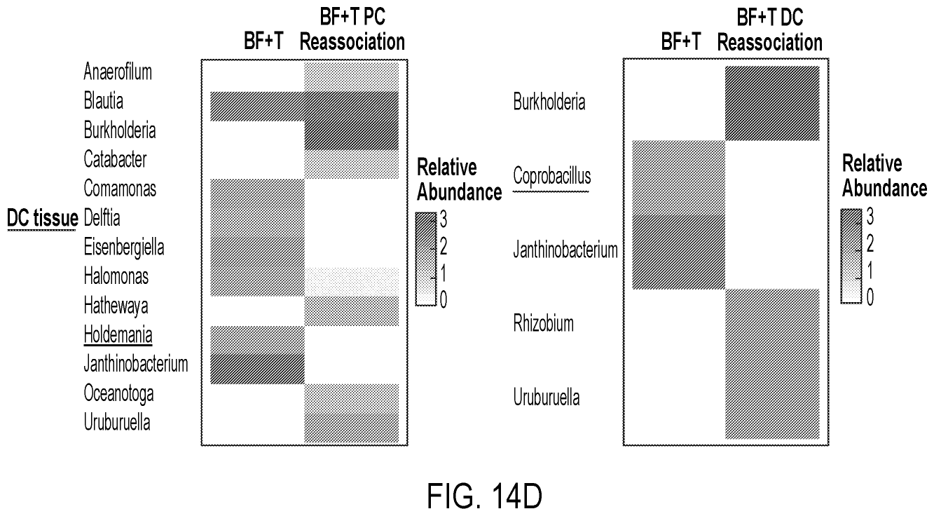

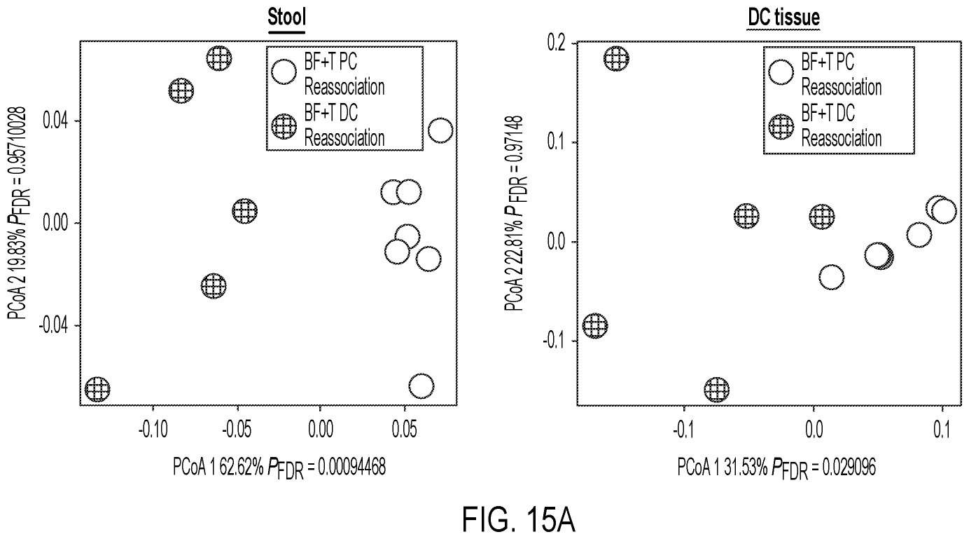

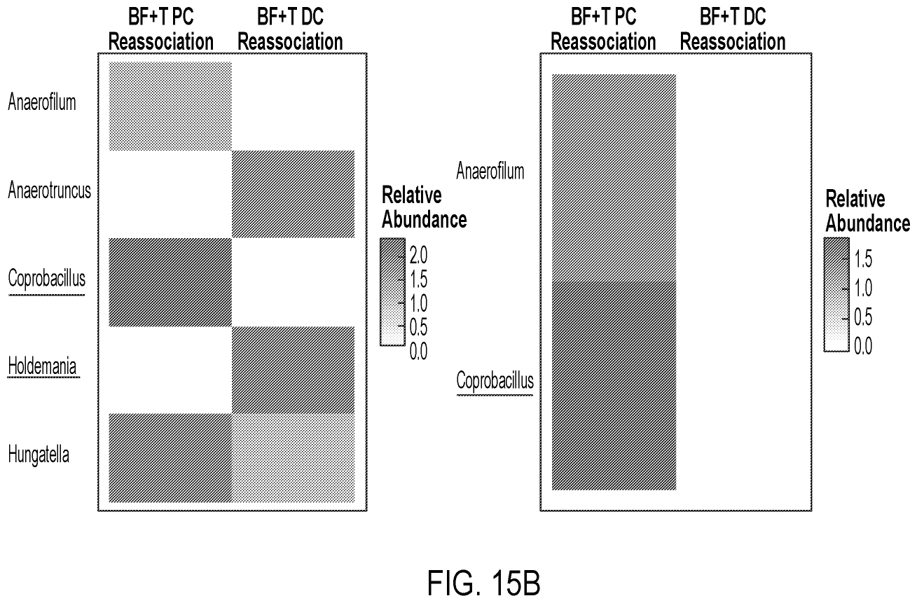

Microbial compositional differences were examined between BF+T and BF-bx reassociated mice. 16S rRNA sequencing revealed separation of stool and distal colon (DC) tissue microbiota from reassociated mice according to the biofilm status of the initial association (FIGS. 9A-9B). Thirteen genera were significantly different (12 genera enriched and one genus depleted) in the stool and/or DC tissue of both the BF+T-associated and reassociated Apc^{MinΔ850/+};Il10^{-/-} mice (FIG. 9C), three of which also correlated with specific miRNAs (FIGS. 8A-8B). Six of these genera (Clostridium XVIII, Erysipelotrichaceae incertae sedis, Escherichia/Shigella, Eubacterium, Parabacteroides, and Robinsoniella) were increased in both the stool and DC tissue of BF+T-associated and reassociated mice compared to BF-bx mice. To determine how much the microbiota composition shifted after reassociation, the microbiotas from the reassociated mice were compared to the initial associated mice, whose tissues were used to generate the inoculums (FIGS. 13A-13D and FIGS. 14A-14D). The BF-bx communities were highly transmissible, with no significant differences based on principal-coordinate analysis (PCoA) of the stool and DC tissue communities (FIGS. 13A-13D). The BF+T communities shifted more after reassociation, with distinct clustering seen in the PCoA (FIGS. 14A-14B). Depending on the colon region the BF+T inoculum was derived from, there were 5 and 13 significantly different genera within the DC tissue or stool compartment; however, only 1 and 5 of these genera were significantly different based on biofilm status in the initial associations (FIGS. 14C-14D). The impact of the location of the colon tissues used to make the BF+T reassociation inoculums on community composition was examined by comparing the stool and DC tissue communities from mice reassociated with PC or DC tissue inoculums (FIG. 15A). It was found that only 2 and 5 genera were significantly different (FIG. 15B) and only 2 of these genera (Coprobacillus and Holdemania) differed according to biofilm status in the initial associations. Out of the 12 genera that were increased in BF+T-associated and reassociated mice, only 1 (Coprobacillus) was not maintained in both groups of BF+T reassociated mice. Thus, regardless of the murine colon region of origin (proximal or distal), the majority of BF+T microbes (11 genera total; Anaerostipes, Clostridium XI, Clostridium XIVa, Clostridium XVIII, Erysipelotrichaceae incertae sedis, Escherichia/Shigella, Eubacterium, Flavonifractor, Lachnospiraceae incertae sedis, Parabacteroides, and Robinsoniella) are able to reestablish and promote cancer when transmitted to a new cohort of GF mice. Taken together, the data indicate there is a core set of bacteria and bacterial gene expression associated with biofilm-positive cancers.

In contrast to previous studies that tested the carcinogenicity of CRC-associated microbiotas by gavaging mice with stools from either CRC or healthy control patients, mucosa-associated BF+T bacteria retain their carcinogenicity when transplanted into a new set of GF mice, regardless of whether they were extracted from the proximal or distal colon. The number of taxa that overlap between the initial association and the reassociation experiments, shown herein (FIGS. 9A-9C), indicate that there is a core, transmissible, cancer-promoting microbiota associated with biofilm-positive cancers.

Four of the BF+T core transmissible cancer-promoting genera overlap with human stool-derived taxa that established and were associated with cancer in mice. Parabacteroides correlated with high tumor numbers in a gnotobiotic azoxymethane (AOM)/dextran sulfate sodium (DSS) C57BL/6 model, and Erysipelotrichaceae, Lachnospiraceae, and a Clostridium XIVa derived from CRC patients were associated with polyp formation in an antibiotic-treated AOM C57BL/6 model. Additionally, increased Erysipelotrichaceae, E. coli, Lachnospiraceae, and Parabacteroides and decreased Bifidobacterium have previously been associated with human CRC patient samples by 16S rRNA gene sequencing studies.

Metagenomic predictions generated from 16S rRNA data identified bacterial secretion systems and motility genes associated with human CRC stool communities and host glycan utilization genes correlated with tumor numbers in human stool-associated AOM/DSS mice. Recent meta-analysis studies of metagenomes from CRC patient fecal samples identified gluconeogenesis, mucin degradation, and colibactin as associated with the CRC microbiome. These bacterial pathways and genes were also increased in BF+T metatranscriptome. PICRUSt analysis of biofilm-positive versus biofilm-negative human CRC tissues also demonstrated an increased sporulation capacity associated with biofilm-positive CRCs contributed by several taxa, among them the Lachnospiraceae family. Similarly, herein, it was shown that genera from the Lachnospiraceae family (Anaerostipes, Clostridium XIVa, Lachnospiracea incertae sedis, and Robinsoniella) and upregulation of multiple sporulation genes in mice transplanted with the BF+T community. There is also overlap between BF+T community gene expression and human periodontitis polymicrobial metatranscriptomes, particularly for genes related to iron acquisition, flagellar synthesis, and the stress response. These findings are interesting since biofilms have also been associated with periodontal disease and F. nucleatum and Porphyromonas spp. are associated with both oral biofilms and colon cancer.

Multiple genes related to nutrient, envelope, DNA damage, and environmental stress responses were increased in the BF+T community that could be indicative of host immune pressures, but could also be associated with a competitive polymicrobial environment, a feature of biofilms. Host iron metabolism changes during inflammation and cancer can promote competition for iron within the intestinal microbiota. Multiple iron acquisition genes, including siderophores and transport receptors, were increased in BF+T mice. The expression of these metabolic and iron acquisition genes could be indicative of a low-nutrient environment, fostering interbacterial competition.

Bacterial adhesion genes are a critical colonization determinant and may also contribute to biofilm formation, in which attachment to host cells represents a key initiating step. There are a number of adhesins expressed in the BF+T community, including type I and IV pili, capsule genes, and proteins that bind to host extracellular matrix (ECM) components such as fibronectin and laminin. On the host side, BF+T mice exhibit upregulation of a laminin subunit and the ECM-degrading matrix metalloproteinase MMP10, indicating alterations to the host ECM. BF+T communities also expressed numerous moonlighting adhesins (such as flagellin, GroEL, DnaK, and elongation factor Tu), putative multifunctional proteins which have been demonstrated in some bacterial strains to bind host cells, mucin, or ECM components. Bacterial adherence has also been identified as an important feature of CRC-associated bacteria, including F. nucleatum and Streptococcus gallolyticus subsp. gallolyticus, the latter of which is capable of forming biofilms on collagen IV, an ECM component. Furthermore, bacteria expressing adhesins that bind to ECM and host glycoproteins may have an additional colonization advantage, as host glycosylation is disrupted during inflammation and cancer with increases in sialylation and fucosylation that can result in decreased host cell adhesion to ECM components. Additionally, some of the effects of CRC-associated bacteria may be contact dependent; for example, colibactin-induced DNA damage requires direct contact between the bacteria and epithelial cells.

Coupled with its role in facilitating colonization and attachment, mucin also represents a source of nutrition for intestinal bacteria. Antibiotic treatment was shown to increase sialic acid levels in the lumen and promoted the expansion of pathogenic bacteria such as C. difficile and Salmonella enterica serovar Typhimurium. Interestingly, sialic acid and other mucin sugar cleavage and transport expression were increased in BF+T mice along with an increased abundance of Clostridium XI and Salmonella. The SusC and SusD outer membrane proteins, involved in oligosaccharide binding and transport, were also increased in the BF+T community (from Bacteroides and Parabacteroides spp.). The upregulation of stress responses, mucin, and other nutrient acquisition genes indicate environmental conditions that could in turn pro-mote virulence expression. Nutrient- and iron-responsive global transcriptional factors such as cyclic AMP receptor protein and ferric uptake regulator (Fur), which were increased in BF+T mice, have also been shown to regulate bacterial virulence expression.

Shotgun metagenomic sequencing of patient stools has revealed that host glycan utilization and virulence factor genes are associated with the CRC micro-biome and that genes in these categories were also overexpressed in the BF+T microbial community. Host inflammation, bacterial iron (Fur), and stress response (Hsp90 chaperone) genes have all been implicated in colibactin regulation, and all of these genes were increased in BF+T mice. Additionally, iron acquisition genes have previously been associated with E. coli mucus colonization, and mucins have the capacity to induce E. coli virulence gene expression. Mucin and its components may also serve as a cue for virulence regulation of other BF+T community members, as they have also been linked to virulence regulation in S. enterica and Campylobacter jejuni. Although the expression of multiple B. fragilis genes were increased in the BF+T community, B. fragilis toxin (bft) was not detected. One possible explanation is that expression of the RprXY two-component system, recently implicated in bft suppression, was significantly increased in BF+T mice. However, even intermittent enterotoxigenic B. fragilis colonization as short as 2 weeks appears to be sufficient to induce tumor formation, so it is possible that bft expression occurred at an earlier time point in the Apc^{MinΔ850/+};Il10^{-/-} model. Taken together, the metatranscriptomic data indicate that the BF+T community expresses more pathogen-related virulence factors and metabolism genes that provide competitive advantages over commensals but may have detrimental side effects to the host.

Members of the Erysipelotrichaceae family, part of the core transmissible bacteria in BF+T mice, were also increased in the microbiota of Western or high-fat diet-fed mice (Clostridium innocuum, Eubacterium dolichum, and Clostridium ramosum) and were associated with increased fat deposition. Diets high in fat and obesity are established risk factors for CRC. Conceivably, the activation of the PPAR signaling pathway within BF+T mice could be a response to colonization with these obesity-associated taxa. PPARs are nuclear hormone receptors that regulate key aspects of lipid and carbohydrate metabolism, including fatty acid synthesis, uptake, and storage, and have both suppressive and promotional effects in CRC.

Branched-chain amino acid (BCAA) biosynthesis was increased in the BF+T community, and serum BCAA have also been associated with metabolic disorders and correlated with intestinal microbiota members such as Bacteroides vulgatus. BCAAs, which include valine, leucine, and isoleucine, may also contribute to fatty acid synthesis, which is also a feature of cancer metabolism. Valine and/or leucine secretion were previously associated with E. coli and polymicrobial environmental biofilms. Polyamines were previously shown through metabolomics to be increased in CRC patients, with a rare polyamine, N1, N12-diacetylspermine detected in biofilm-positive CRCs, and we found that multiple microbial polyamine-related genes were in-creased in BF+T mice. Polyamines increase in proliferating cells and can promote tumor growth and invasion and are also important to bacterial biofilm formation. Several transporter genes (Slc22a3, Abcb1a, and Abcb1b) that have been previously implicated in polyamine uptake were upregulated in BF+T mice. Thus, biofilm-associated communities and their associated metabolism pathways have the potential to modulate host metabolism, which may promote cancer.

In addition to bacterial components and metabolites, miRNAs represent another mode of host-microbe interplay during cancer. Profiling the fecal miRNAs of Apc^{MinΔ850/+};Il10^{-/-} mice under different microbial conditions allowed us to identify specific miRNAs that were associated with biofilm/CRC status. A few of the CRC-associated miRNAs that were identified have conserved sequences with human miRNAs (hsa-miR-21-5p, hsa-miR-142-5p, and hsa-miR-146a-5p) that are increased in CRC patients. Mmu-miR-21a-5p was significantly increased in the BF+T mice, and F. nucleatum has previously been shown to increase miR-21, indicating that miR-21 may be targeted by multiple CRC-associated bacterial genera.

Although the depleted miRNAs in BF+T mice, miR-690 and miR-709, are not found in humans, they do share several CRC-related gene targets (such as Ctnnb1, Il6ra, Stat3, Src, and Zeb1) with other miRNAs depleted in CRC. Though none of these genes were significantly DE according to biofilm status in the mouse colon tissue RNA-seq data set, it is possible the luminal miRNAs might target other regions of the colon or specific intestinal epithelial cell types. Additionally, even though miRNAs have been shown to primarily control gene expression through mRNA degradation, translational repression is also possible. Alternatively, another mechanism of miRNA regulation could relate to targeting the RNA-induced silencing complex genes like Argo1, Argo2, Argo3, Argo4, Cnot6, and Dcp2, which are predicted targets of multiple significant DE miRNAs, the majority of which are increased in BF+T mice.

miRNAs also have the capacity to target bacterial genes and impact microbial composition, and the computational predictions indicate that newly discovered miRNAs (those with higher numbers in their names) preferentially target bacterial genes. These miRNAs include miR-2137, miR-5126, miR-6239, miR-6240, and miR-6538, which were also increased in DSS-treated mice, where microbiota composition also contributes to disease susceptibility. Many of these miRNAs were predicted to have redundant bacterial targets (including genes regulating motility, secretion, outer membrane proteins, stress response, iron acquisition, and carbohydrate utilization/transport) that overlap with genes that were increased in the BF+T microbial community. miR-6239 and -6240 were decreased in BF+T mice, but miR-2137, -5126, and -6538 were increased in mice, regardless of biofilm status.

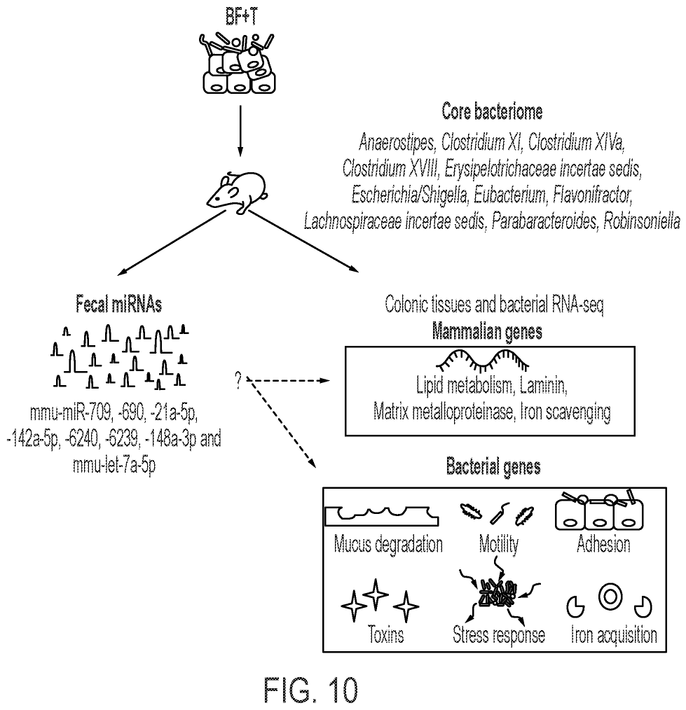

Without wishing to be bound by theory, the data indicate a complex interplay between BF+T-associated bacteria, their gene expression, the host transcriptome, and miRNAs that may contribute to CRC pathogenesis (FIG. 10).

### Materials and Methods

### Animals.

Germfree (GF) 129/SvEv Apc^{MinΔ}850/+;Il10^{-/-} mice were transferred to separate gnotobiotic experimental isolators based on inoculum type for the duration of the association.

Initial Associations with Human Tissue-Associated Microbes.

GF 129/Apc^{MinΔ}850/+;Il10^{-/-} mice were inoculated with pooled tissue-derived microbes from biofilm-negative tissues collected from healthy patients via colonoscopy biopsy (BF-bx) or biofilm-positive tumor tissues collected from CRC patients (BF+T) via surgical resection. Patient tissues were collected and screened for biofilm status via fluorescence in situ hybridization (FISH) (biofilm-positive criteria, polymicrobial, within the mucus layer, spanning 200 μm, and >10⁹ bacteria/ml) as described previously. Tissues were analyzed with the universal bacterial probe (EUB338), and a nonsense probe (non338) was used as a negative control. Additional FISH analysis was conducted on biofilm-positive tumor tissues with probes to detect Bacteroidetes, Lachnospiraceae, Fusobacteria, and Proteobacteria. The probe sequences are listed in Table S4 in reference 12. Each inoculum was prepared anaerobically by homogenizing tissue (tissue pooled from five different patients) in phosphate-buffered saline (PBS), and FISH images for these tissues can be found in Fig. S1 in reference 13. Each mouse received 100 to 200 μl of inoculum, and associations were carried out for 12 to 20 weeks (FIG. 5A I). Tissues and/or stools from mice collected 12 weeks after association were used for transcriptome sequencing (RNA-seq), microRNA sequencing (miRNA-seq), and 16S rRNA gene sequencing analyses (FIG. 5A II).

### Mouse Reassociation Inoculums.

Mouse reassociation inoculums (FIG. 5A III) were made from colon tissues from 12-week BF-bx-associated (cohort 2) and 16- to 20-week BF+T-associated (cohort 3) Apc^{MinΔ850/+};Il10^{-/-} mice. After the colon was flushed 1× with PBS, tissue snips were taken from both the distal colon (DC) and proximal colon (PC) and stored at −80° C. until time of inoculum preparation. Each inoculum was prepared from colon tissue snips pooled from four mice. All inoculums were prepared anaerobically by mincing and homogenizing tissue snips in prereduced PBS. The BF-bx reassociation inoculum was prepared from inflamed (average inflammation score of 2.5/4) distal colon tissues (BF-bx DC). The two BF+T reassociation inoculums were prepared from mostly normal (average PC inflammation score of 0.9/4) proximal colon tissues (BF+T PC) or distal colon tissues (BF+T DC) from the same four mice with colitis and tumors (average DC inflammation score of 3.6/4; average number of tumors=5.5, range=3 to 10 tumors).

Reassociation with Mouse Tissue-Associated Microbes.

Six- to 13-week GF 129/SvEv Apc^{MinΔ850/+}; Il10^{-/-} (males and females) were transferred to gnotobiotic isolators (separate isolator for each experimental group) and gavaged with 100 to 200 μl of inoculum (FIG. 5A III). Mice were euthanized after 12 weeks, and the colon was flushed once with PBS and then cut and splayed longitudinally for macroscopic tumor counts. About 2×0.5 cm tissue snips from the proximal and distal colon were collected by flash freezing in liquid nitrogen and stored at −80° C. until analysis.

### Stool and Distal Colon Tissue DNA Extraction.

Stool and DC tissue DNA (FIGS. 5A II and IV) was extracted via phenol-chloroform separation by lysing the cells with phenol-chloroform-isoamyl alcohol (25:24:1) and 0.1-mm zirconium glass beads on a bead beater (Precellys), followed by phase separation with chloroform-isoamyl alcohol (24:1), DNA precipitation with ethanol, and subsequent purification with the DNeasy Blood & Tissue kit (Qiagen catalog no. 69506).

16S rRNA Sequencing.

The 16S rRNA V1-V3 hypervariable region was amplified using barcoded primer pairs 27F (5=-AGAGTTTGATCCTGGCTCAG-3=) and 534R (5=-ATTACCGCGGCTGCTGG-3=) with universal Illumina paired-end adapter sequences. PCR products were purified, quantified, and pooled as described previously and sequenced with an Illumina MiSeq (as two separate runs). The first 16S rRNA run (Run01, FIG. 5A II) included stool and DC tissue samples from BF-bx cohort 1, BF+T cohorts 1 to 3, and two different biofilm-positive groups. Differentially abundant taxa were identified by comparing stool (1- and 12-week time points) and DC tissue (12-week time point) communities collected from BF-bx #1, BF+T #1-2, and two additional biofilm-positive groups reported in Tomkovich et al. The second 16S rRNA run (Run02, FIGS. 5A II and IV) included stool and DC tissue samples from BF-bx cohort 2 and the three reassociation groups (BF-bx DC, BF+T PC, and BF+T DC). Comparisons between initial associations and reassociations were assessed by comparing the microbiota from mice whose tissues were used for the inoculums (BF-bx #2 and BF+T #3) to the reassociation microbiotas (BF-bx DC, BF+T PC, and BF+T DC).

16S rRNA Sequencing Analysis.

Reads were preprocessed using Quantitative Insights into Microbial Ecology (QIIME) version 1.9.1 including trimming and filtering at Q20. The final set of reads was fed to the RDP (Ribosomal Database Project) classifier version 2.12 with the confidence set at 80%. Reads were grouped by genera, and samples with less than 1,000 total reads and genera with less than 5 reads were removed. The resulting counts were normalized and log 10 transformed using the following formula:

$${log}_{10} ( \frac{R C}{n} \times \frac{\sum x}{N} + 1 )$$

where RC is the read count for a particular taxon in a particular sample, n is the total number of reads in that sample, the sum of x is the total number of reads in all samples, and N is the total number of samples. The principal-coordinate analysis (PCoA) was generated from the Bray-Curtis distance of the normalized and log 10-transformed counts using the phyloseq R package.

Genera significant for biofilm group (BF-bx, BF+T, BF-bx DC, BF+T PC, and BF+T DC) were detected using the lme function in the R nlme package, with the REML method to fit a mixed linear model of the form:

geus˜variable+1|cage+ε

where genus indicates the log 10 normalized abundance of a particular genera, variable indicates either the biofilm group or PCoA axis, and 11cage indicates that the cage was used as a random effect. Then, an analysis of variance (ANOVA) was run on the above model to generate P values for biofilm group or PCoA axis. Possible cage effect was checked by comparing the above model and a model with the cage removed (genus˜variable+ε) using ANOVA. The P values were adjusted for multiple hypothesis testing in R using the p.adjust function employing the method of Benjamini and Hochberg. The heatmaps were generated using the R function ggplot2.

Two additional analyses were performed on the 16S rRNA data, the first utilizing QIIME v. 1.9.1 closed-reference at 97% similarity level using the Greengenes reference data set release 13_8 and the second employing Deblur workflow v. 1.0.3 with the default parameters (using Deblur's default positive and negative reference filtering) and trim length set to 100 bases. Both pipelines showed no significant separation between the BF-bx and BF-bx DC samples (see FIGS. 13C and D).

RNA Extraction, rRNA Depletion, and RNA Sequencing.

Total RNA was extracted from frozen proximal colon tissue snips (FIG. 5A II) using the mirVana miRNA isolation kit, with phenol (ThermoFisher Scientific catalog no. AM1560) according to the manufacturer's instructions, with the addition of an ˜1:1 mix of 1-mm acid-washed glass beads and 0.1-mm zirconia beads and a Precellys24 (Bertin Instruments catalog no. EQ03119-200-RD000.0) bead beater for tissue disruption and lysis. Extracted RNA was treated with the Turbo DNA-free kit (ThermoFisher Scientific catalog no. AM1907) to remove DNA. Quality control, rRNA depletion, and cDNA library preparation were performed by the University of Florida's Interdisciplinary Center for Biotechnology Research (ICBR) Gene Expression and Genotyping core using the Agilent 2100 bioanalyzer (Agilent Genomics catalog no. G2939BA), Ribo-Zero Gold rRNA removal kit (Epidemiology) (Illumina catalog no. MRZE724) and ScriptSeq v2 RNASeq library preparation kit (Illumina catalog no. SSV21124) starting with 1 μg total RNA. Samples were sequenced by the University of Florida ICBR NextGen DNA Sequencing core on the Illumina HiSeq 3000 (2×100 run), multiplexing each sample into three lanes to avoid lane effect.

### Mouse RNA-Seq Analysis.

Reads were quality filtered at Q20 and trimmed to remove remaining adapters using Trimmomatic version 0.36. The resulting reads were aligned to Illumina iGenome Mus musculus Ensembl GRCm38 reference genome using Tophat version 2.1.1 utilizing Bowtie2 version 2.3.0 following the approach of Gilad and Mizrahi-Man. The resulting alignments (averaging 34,079,158 concordant read pairs per sample) were processed using Cufflinks version 2.2.1 along with Illumina iGenome Mus musculus Ensembl GRCm38 gene transfer format file, after masking rRNA features. Cuffquant was used to perform transcript quantification and exported the raw counts (nonnormalized counts) to text files. The raw counts were then imported to edgeR version 3.16.5 for detecting differentially expressed (DE) genes. A gene was considered for the differential expression test if it was present in at least 50% of the samples. Agene DE was considered if its edgeR false-discovery rate (FDR)-adjusted P value (PFDR) was <0.05. Parallel analysis using feature Counts from the subread package version 1.5.3 for transcript quantification showed similar results (data not shown). Pathway analysis was conducted through GAGE version 2.24 using Mus musculus (mmu) Kyoto Encyclopedia of Genes and Genomes (KEGG) pathways, and genes were mapped to KEGG pathways using Pathview. A pathway was considered significant if its GAGE false-discovery rate (q value) was less than 0.05. The effect of sequencing lane on the clustering of the samples was tested (FIG. 6A) and found it to be insignificant (P value>0.05) (data not shown).

TABLE 2
Ratios of DE genes in BF + T and BF-bx groups

| | % of genes | | |
|---|---|---|---|
| | DE genes | Upregulated genes | Downregulated genes |
| | (FDR < 0.05) | In BF + T group | In BF + T group |
| | (% of input | (% of DE | (% of DE |
| Data set | transcripts) | transcripts) | transcripts) |
| Complete | 36 | 93 | 7 |
| Rarefied^{a} | 40 | 88 | 12 |

^{a}The rarefied data set was based on 140,000 reads per sample.

### Metatranscriptome Analysis.

Quality-filtered and trimmed reads from above were aligned to iGenome Mus musculus Ensembl GRCm38 reference genome using BWA version 0.7.16a, and reads with alignments were excluded from further analysis. The remaining reads were then filtered from rRNA and tRNA sequences by aligning (using BWA) to a collection of NCBI rRNA and tRNA sequences and SILVA database sequences, resulting in an average of 1,208,429 reads per sample, which were then submitted for de novo assembly using Trinity version 2.4.0. The resulting assembly was annotated using Trinotate version 3.0.1 (trinotate.github.io) with the following databases: uniprot_sprot, Pfam, and Virulence Factor Database (VFDB). The resulting annotations were examined, and sequences annotated as nonbac-terial were removed. Transcript abundance was determined using RNA-seq by expectation maximization (RSEM) through Trinity's align_and_estimate_abundance.pl script, and the counts were imported to edgeR version 3.16.5 for differential expression analysis. A gene was considered for the differential expression test if it was present in at least 50% of the samples. Transcript DE was considered if its edgeR FDR-adjusted P value was <0.05. To account for normalization artifacts, the ratios of DE genes between the BF+T and BF-bx groups generated from a rarefied data set that was based on 140,000 reads per sample were examined (Table 2). The similar ratios of DE genes from the complete and rarefied data sets indicate that findings are not an artifact of normalization. A second analysis (reference-based analysis) was conducted by aligning the reads submitted for de novo assembly to the human gut microbiome integrated gene catalog (IGC) using Bowtie2 (v.2.3.4.2) followed by quantification using featureCounts from the subread package (v.1.5.3) and obtained similar results (FIG. 11A).

Pathway analysis was conducted through GAGE version 2.24 using Kyoto Encyclopedia of Genes and Genomes (KEGG) reference pathways on the assembled transcript and The Human Microbiome Project (HMP) Unified Metabolic Analysis Network (HUMAnN) on the unassembled reads. Genes were mapped to KEGG pathways using Pathview. A pathway was considered significant if its q value was <0.05. Weighted gene coexpression network analysis (WGCNA) version 1.68 was utilized to detect modules in each biofilm status samples using the blockwiseConsensusModules function which performs the network construction and consensus module detection. The hub gene in each detected module was identified using the WGCNA function chooseTopHubInEachModule. The sequencing lane had no effect on the clustering of the samples in FIG. 5A (P value>0.05, data not shown).

miRNA Extraction and Sequencing.

Small RNAs were extracted from snap-frozen stool samples (FIG. 5A II) using the mirVana miRNA isolation kit. Because of the low amount of small RNA, GF stools were pooled from 13 Apc^{MinΔ850/+};Il10^{-/-} mice (20- to 44-week age range) total, or stools from 2 to 5 mice per sample (n=4). BF-bx and BF+T small RNAs were extracted from the stools of 12-week-associated BF-bx (n=7) and BF+T(n=10) Apc^{MinΔ850/+};Il10^{-/-} mice. cDNA libraries were synthesized with the NEBNext Multiplex Small RNA Library Prep Set for Illumina kit (New England Biolabs catalog no. E7300) and small RNAs for each library (21- to 30-nucleotide size range) were gel purified. For the GF, BF-bx, and BF+T comparisons, a pool of 21 libraries (equivalent molar concentrations; 4 GF, 7 BF-bx, and 10 BF+T) were multiplexed and sequenced using the Illumina Miseq.

miRNA Analysis.

CAP-miRSeq was used to process the miRNA sequences. The databases and reference sequences that ship with CAP-miRSeq were used for all the analyses. Briefly, sequences were filtered and trimmed using cutadapt. Quantification of miRNA was done using miRDeep2, and DE miRNAs were detected using edgeR version 3.16.5. An miRNA DE was considered if its edgeR FDR-adjusted P value was <0.05. Principal-component analysis (PCA) was created using R's prcomp function from the normalized and log 10-transformed miRNA counts according to the equation above.

Correlations with microbiota taxon abundance were done using R lm function, P values were determined using R's ANOVA function, and FDR correction was done using R's p.adjust function employing the method of Benjamini and Hochberg and only those with a FDR-adjusted P value of <0.05 were considered.

Mouse miRNA targets were predicted using miRDB, and bacterial target prediction for mice miRNA was done using PITA on the assembled bacterial transcripts from the RNA-seq data described above. A bacterial transcript was considered a potential target for a particular mouse miRNA if its ΔΔG score was less than or equal to −15 kcal/mol.

For miRNA expression correlation with tumor numbers, two BF-bx samples were excluded because they were fixed without splaying the colon so tumor counts could not be generated. Correlation was done using Spearman's rank correlation through R's cor.test function.

### Statistical Analyses.

For all sequencing analyses, a taxon or miRNA was considered only if it was present in at least 30% of the comparison samples, and statistics are described in above 16S rRNA sequencing, mouse RNA-seq, metatranscriptome and miRNA analysis sections. P values of <0.05 were considered statistically significant.

### Example 3: Knockdown of Endogenous Bacterial Gene with EV-Loaded siRNA

This example describes use of EV-loaded siRNA to silence endogenous bacterial gene expression. HCT-116 cells were lipofected with an siRNA sequence targeting the bacterial FliC mRNA, which encodes a flagellar protein essential for virulence of certain bacteria (e.g., E. coli). EVs released by the cultured cells were isolated by ultracentrifugation. The FliC siRNA sequence contained in the EVs is complimentary to E. coli NC101 FliC mRNA, and contains one nucleotide that creates a G:U (guanidine:uridine) wobble base pair interaction between the guide strand of the siRNA and the FliC mRNA.

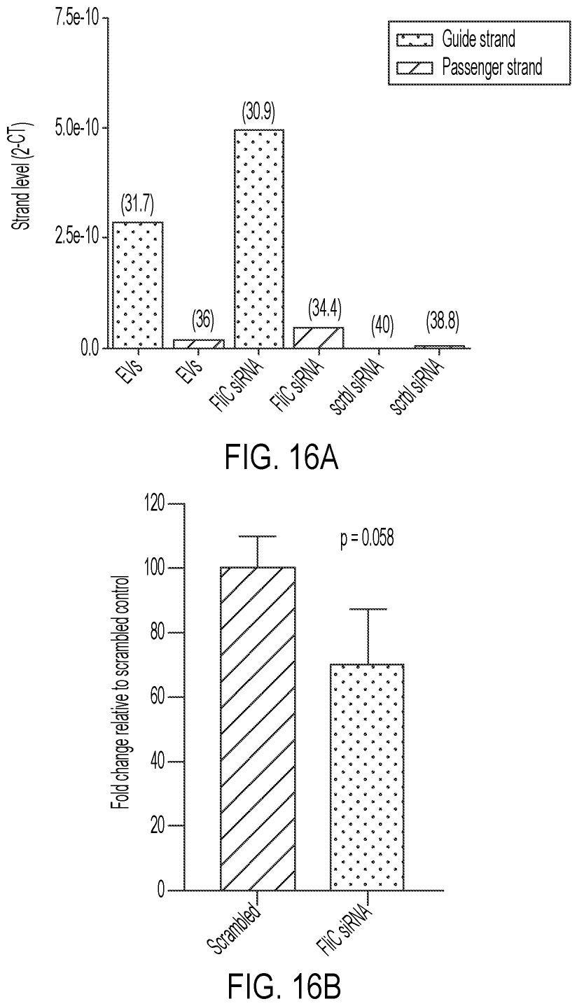

HCT-116 cell EVs were exposed to NC101 E. coli (100 EVs per CFU of E. coli) for three hours. RNA was isolated from the EV or the E. coli and the amount of active (guide) strand or inactive (passenger) strand was determined by qRT-PCR. Data indicate that FliC siRNA was properly processed to produce siRNA guide strands in both EVs and E. coli (FIG. 16A). FliC siRNA loaded into HCT-116 cell EVs was exposed to NC101 bacteria (100 EVs per CFU of E. coli) for three hours and then RNA was isolated from the bacteria and the amount of FliC mRNA normalized to 16S rRNA and relative to scrambled control oligo was determined by qRT-PCR. Data indicate that EV-delivered FliC siRNA inhibited FliC mRNA expression relative to scrambled control.

### EQUIVALENTS

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method of delivering one or more inhibitory nucleic acids to a prokaryotic cell, comprising contacting the prokaryotic cell with an extracellular vesicle (EV) comprising the one or more inhibitory nucleic acids, wherein the one or more inhibitory nucleic acids target (e.g., hybridize to) one or more genes in the prokaryotic cell, and wherein the EV is derived from a mammalian cell.

2. A method of regulating the expression of one or more genes in a prokaryotic cell, comprising contacting the prokaryotic cell with an extracellular vesicle (EV) comprising one or more inhibitory nucleic acids that target the one or more genes, wherein the EV is derived from a mammalian cell.

3. A method of treating a disease in a subject in need thereof, comprising administering to the subject isolated extracellular vesicles (EVs) comprising one or more inhibitory nucleic acids that target one or more genes in a prokaryotic cell, and wherein the isolated EVs are derived from a mammalian cell.

4. The method of claim 3, wherein the subject is a human.

5. The method of claim 3 or 4, wherein the disease is a metabolic disorder, a cardiovascular disease, cancer, an autoimmune disease, or an inflammatory disease.

6. The method of claim 3 or 4, wherein the disease is metabolic syndrome, inflammatory bowel disease, hypertension, asthma, diabetes, celiac disease, obesity, non-alcoholic fatty liver disease, non-alcoholic steatohepatitis, or rheumatoid arthritis.

7. The method of claim 5, wherein the disease is a cancer selected from: lymphoma, leukemia, multiple myeloma, breast cancer, prostate cancer, esophageal cancer, stomach cancer, colorectal cancer, liver cancer, cervical cancer, ovarian or uterine cancer, pancreatic cancer, lung cancer, brain cancer, sarcoma, and skin cancer.

8. The method of claim 3 or 4, wherein the disease is a bacterial infection.

9. A method of modifying the composition of the microbiota in a subject, comprising administering to the subject isolated extracellular vesicles (EVs) comprising one or more inhibitory nucleic acids that target one or more genes in a prokaryotic cell, and wherein the isolated EVs are derived from a mammalian cell.

10. The method of claim 9, wherein the method comprises killing prokaryotic cells of a species.

11. The method of claim 9 or 10, wherein the subject is a human.

12. The method of any one of claims 9-11, wherein the microbiota is gastrointestinal microbiota, mucosal microbiota, skin microbiota, microbiota of the respiratory system, microbiota of the ear, nose, and throat, oral microbiota, or microbiota of the urinary tract.

13. The method of any preceding claim, wherein the EV is an exosome or microvesicle.

14. The method of any preceding claim, wherein the one or more inhibitory nucleic acids are small interfering RNA (siRNA), microRNA (miRNA), short hairpin RNA (shRNA), antisense RNA, dsRNA, artificial miRNA, circular RNA, long non-coding RNA (lncRNA), or piwi-interacting RNA (piRNA) molecules.

15. The method of any preceding claim, wherein the EV further comprises one or more components of an RNA-induced silencing complex (RISC).

16. The method of claim 15, wherein the EV further comprises one or more of protein kinase RNA activator (PACT), transactivation response RNA binding protein (TRBP), Dicer, Argonaute, Drosha, and Pasha.

17. The method of any preceding claim, wherein the target gene is located chromosomally.

18. The method of any preceding claim, wherein the target gene is located episomally.

19. The method of any preceding claim, wherein the prokaryotic cell is a bacterial cell.

20. The method of claim 19, wherein the bacterial cell is a pathogenic bacterium.

21. The method of claim 19, wherein the bacterial cell is a commensal bacterium.

22. The method of claim 19, wherein the bacterial cell is a cell type found in human microbiota.

23. The method of claim 19, wherein the bacterial cell is of the genus Actinomyces, Akkermansia, Alistipes, Anaerofilum, Anaerostipes, Bacteroides, Barnesiella, Bifidobacterium, Blautia, Catabacter, Clostridium, Coprobacillus, Enterobacter, Enterococcus, Erysipelotrichaceae, Escherichia, Eubacterium, Faecalibacterium, Flavinofractor, Flavobacterium, Fusobacterium, Gordonibacter, Haemophilus, Holdemania, Hungatella, Lachnospiracea, Lactobacillus, Parabacteroides, Phascolarctobacterium, Prevotella, Pseudomonas, Robinsoniella, Romboutsia, Roseburia, Ruminococcus, Salmonella, Shigella, or Terrisporobacter.

24. The method of any preceding claim, wherein the mammalian cell is an amniocyte cell, a cardiac progenitor cell, a cardiomyocyte, an epidermal cell, an epithelial cell, a fibroblast, a hematopoietic stem cell, a mesenchymal stem cell, a neuronal precursor cell, a neuron, a platelet, or a reticulocyte.

25. The method of claim 24, wherein the mesenchymal stem cell is derived from adipocytes, neurons, bone marrow, or umbilical cord.

26. The method of any one of claims 1-23, wherein the mammalian cell is a cell line selected from: HEK-293, HEK-293T, CHO, PERC6, BJ, fHDF/TERT166, AGE1.HN, CAP, and RPTEC/TERT1.

27. The method of any one of claims 1-23, wherein the mammalian cell is a cancer cell.

28. The method of claim 27, wherein the cancer cell is a bladder cancer cell line, a breast cancer cell line, a brain cancer cell line, a colorectal cancer cell line, a head and neck cancer cell line, a leukemia cell line, a liver cancer cell line, a lung cancer cell line, a lymphoma cell line, an ovarian cancer cell line, a pancreatic cancer cell line, a sarcoma cell line, a stomach cancer cell line, or a uterine cancer cell line.

29. The method of claim 27, wherein the cancer cell is a cell line selected from: HT-1080, HeLa, HT29, HTC116, MBA-MB-231, MCF7, Panc-1, OVCAR-4, SW620, KM12, Colo205, and HCT-15.

30. The method of any preceding claim, wherein the one or more genes encode a gene product associated with antibiotic resistance, virulence, biofilm formation, stress response, protein export, bacterial secretion, amino acid biosynthesis, metabolic pathways, flagellar assembly, carbon fixation, adhesion, or iron acquisition.

31. The method of any preceding claim, wherein the one or more genes encode a toxin, an adhesin, a receptor, a membrane protein, a structural protein, or a secreted protein.

32. The method of any preceding claim, wherein the EV derived from the mammalian cell is prepared by a method comprising:
(a) introducing a nucleic acid encoding or comprising the inhibitory nucleic acid into the mammalian cell;
(b) culturing the mammalian cell under conditions under which the mammalian cell produces EVs; and
(c) isolating the EV from the mammalian cell.

33. A method of preparing extracellular vesicles (EVs) derived from a mammalian cell, comprising:
(a) introducing one or more nucleic acids comprising or encoding one or more inhibitory nucleic acids that target one or more prokaryotic genes, into a mammalian cell;
(b) culturing the mammalian cell under conditions under which the mammalian cell produces EVs; and
(c) isolating the EVs from the mammalian cell.

34. The method of claim 33, further comprising purifying the EVs.

35. The method of claim 33 or 34, wherein the EVs are exosomes or microvesicles.

36. The method of any one of claims 33-35, wherein the method further comprises replacing the culture medium with a serum-free medium to increase EV production in step (b).

37. The method of any one of claims 33-36, wherein the prokaryote is a bacterium.

38. The method of any one of claims 33-37, wherein the mammalian cell is an amniocyte cell, a cardiac progenitor cell, a cardiomyocyte, an epidermal cell, an epithelial cell, a fibroblast, a hematopoietic stem cell, a mesenchymal stem cell, a neuronal precursor cell, a neuron, a platelet, or a reticulocyte.

39. The method of claim 38, wherein the mesenchymal stem cell is derived from adipocytes, neurons, bone marrow, or umbilical cord.

40. The method of any one of claims 33-37, wherein the mammalian cell is a cell line selected from: HEK-293, HEK-293T, CHO, PERC6, BJ, fHDF/TERT166, AGE1.HN, CAP, and RPTEC/TERT1.

41. The method of any one of claims 33-37, wherein the mammalian cell is a cancer cell.

42. The method of claim 41, wherein the cancer cell is a bladder cancer cell line, a breast cancer cell line, a brain cancer cell line, a colorectal cancer cell line, a head and neck cancer cell line, a leukemia cell line, a liver cancer cell line, a lung cancer cell line, a lymphoma cell line, an ovarian cancer cell line, a pancreatic cancer cell line, a sarcoma cell line, a stomach cancer cell line, a uterine cancer cell line.

43. The method of claim 41, wherein the cancer cell is a cell line selected from: HT-1080, HeLa, HT29, HTC116, MBA-MB-231, MCF7, Panc-1, OVCAR-4, SW620, KM12, Colo205, and HCT-15.

44. The method of any one of claims 33-43, wherein the one or more nucleic acids encoding or comprising the one or more inhibitory nucleic acids are introduced by electroporation, transfection, gene gun, direct injection, microinjection, nucleofection, lipofection, or high-pressure spraying.

45. The method of any one of claims 33-44, wherein the one or more genes encode a gene product associated with antibiotic resistance, virulence, biofilm formation, stress response, protein export, bacterial secretion, amino acid biosynthesis, metabolic pathways, flagellar assembly, carbon fixation, adhesion, or iron acquisition.

46. The method of any one of claims 33-45, wherein the one or more genes encode a toxin, an adhesin, a receptor, a membrane protein, a structural protein, or a secreted protein.

47. A composition comprising EVs prepared by the method of any one of claims 33-46.

48. A prokaryotic cell comprising an EV prepared by the method of any one of claims 33-46.

49. The prokaryotic cell of claim 48, wherein the cell is in a subject or in an organ of a subject.

50. The prokaryotic cell of claim 48 or 49, wherein the cell is a bacterial cell.

51. A prokaryotic cell comprising an inhibitory nucleic acid derived from a mammalian cell and one or more components of an RNA-induced silencing complex (RISC).

52. The prokaryotic cell of claim 51, further comprising one or more of protein kinase RNA activator (PACT), transactivation response RNA binding protein (TRBP), Dicer, Argonaute, Drosha, and Pasha.

53. The prokaryotic cell of claim 51 or 52, wherein the inhibitory nucleic acid and the one or more components of a RISC are present within an extracellular vesicle (EV) derived from a mammalian cell.

54. The prokaryotic cell of claim 53, wherein the EV is an exosome.

55. The prokaryotic cell of claim 54, wherein the exosome comprises one or more polypeptides selected from: Alix, TSG101, CD9, CD63, CD81, CD82, Flotillin-1, CD24, HSC70, HSP90, ACTB, GAPDH, ENO1, YWHAZ, and PKM2.

56. The prokaryotic cell of any one of claims 51-55, wherein the inhibitory nucleic acid targets a gene product associated with antibiotic resistance, virulence, biofilm formation, stress response, protein export, bacterial secretion, amino acid biosynthesis, metabolic pathways, flagellar assembly, carbon fixation, adhesion, or iron acquisition.

57. The prokaryotic cell of any one of claims 51-56, wherein the inhibitory nucleic acid targets a gene encoding a toxin, an adhesin, a receptor, a membrane protein, a structural protein, or a secreted protein.
